SEQUENCE LISTING (9 sequences)

SEQ ID NO: 1 - DNA, 20 bp, synthetic construct
  misc_feature 1..20: note = Primer
  source 1..20: mol_type = other DNA
ggagttcaagaaggagtttc

SEQ ID NO: 2 - DNA, 19 bp, synthetic construct
  misc_feature 1..19: note = Primer
  source 1..19: mol_type = other DNA
ccacatacagtgtctggta

SEQ ID NO: 3 - DNA, 20 bp, synthetic construct
  misc_feature 1..20: note = Probe
  misc_feature 1: note = /note="5'FAM"
  misc_feature 20: note = /note="3'BHQ1"
  source 1..20: mol_type = other DNA
ccgcttacgccagaggaact

SEQ ID NO: 4 - DNA, 20 bp, synthetic construct
  misc_feature 1..20: note = Primer
  source 1..20: mol_type = other DNA
agggatttgaatcatgtttg

SEQ ID NO: 5 - DNA, 19 bp, synthetic construct
  misc_feature 1..19: note = Primer
  source 1..19: mol_type = other DNA
ctgctagttctttactggc

SEQ ID NO: 6 - DNA, 23 bp, synthetic construct
  misc_feature 1..23: note = Probe
  misc_feature 1: note = /note="5'HEX"
  misc_feature 23: note = /note="3'BHQ1"
  source 1..23: mol_type = other DNA
tgtagattcaacttgccgctgtc

SEQ ID NO: 7 - DNA, 18 bp, synthetic construct
  misc_feature 1..18: note = Primer
  source 1..18: mol_type = other DNA
ctggaccaaacacaaatg

SEQ ID NO: 8 - DNA, 18 bp, synthetic construct
  misc_feature 1..18: note = Primer
  source 1..18: mol_type = other DNA
atgccttctttcaccttc

SEQ ID NO: 9 - DNA, 20 bp, synthetic construct
  misc_feature 1..20: note = Probe
  misc_feature 1: note = /note="5'Texas red"
  misc_feature 20: note = /note="3'BHQ2"
  source 1..20: mol_type = other DNA
ttgccatccagccactcagt

(19) United States
(12) Patent Application Publication
(10) Pub. No.: US 2025/0282732 A1
(43) Pub. Date: Sep. 11, 2025

(54) NOVEL ESTERS OF 1,2,3,4,4a,5,6,7,8,9,10,10a-DODECAHYDROBENZO[G]QUINOLIN-6-OL COMPOUNDS AND USES THEREOF

(71) Applicant: INTEGRATIVE RESEARCH LABORATORIES SWEDEN AB, Göteborg (SE)

(72) Inventors: Sverker Von Unge, Göteborg (SE); Clas Sonesson, Billdal (SE)

(21) Appl. No.: 18/858,955

(22) PCT Filed: Apr. 24, 2023
(86) PCT No.: PCT/EP2023/060703

§ 371 Date: Oct. 22, 2024

(30) Foreign Application Priority Data
Apr. 25, 2022 (SE) 2250493-0
Jun. 23, 2022 (SE) 2250792-5

(51) Int. Cl. C07D 221/08; A61K 31/473

(52) U.S. Cl. CPC C07D 221/08; A61K 31/473

(57) ABSTRACT

There is disclosed a compound of Formula (III), a method for manufacturing thereof as well as uses thereof.

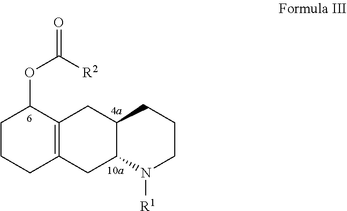

### TECHNICAL FIELD

The present disclosure relates to novel esters of 1,2,3,4,4a,5,6,7,8,9,10,10a-dodecahydrobenzo[g]quinolin-6-ol compounds, a method for preparing said compounds, a pharmaceutical composition comprising said compounds and to uses of said compounds.

### BACKGROUND

Neurodegenerative diseases and neurological disorders are becoming increasingly prevalent with the growing number of aging populations worldwide. One of the most common of these diseases and disorders is Parkinson's disease which is characterized by tremors, motor disturbances and coordination defects. Parkinson's disease is believed to be caused by deterioration of dopamine-producing neurons of the brain, in particular the substantia nigra neurons.

Currently there is no known cure for Parkinson's disease. Instead, the treatment of Parkinson's disease is focused on providing symptom relief.

The state of the art treatment of Parkinson's disease involves administering to the patient L-dopa or apomorphine. These compounds are known to exert their action by being agonists of the D1 and/or D2 dopamine receptors. In the case of L-dopa, it is its active metabolite dopamine that is the species that interacts at the D1 and/or D2 dopamine receptors. However, therapies involving L-dopa or apomorphine are associated with drawbacks. For instance, L-dopa has low and variable bioavailability which depends on protein intake. Further, use of L-dopa may result in long term complications such as dyskinesias. Apomorphine has a very short duration of action and a patient therefore has to take multiple injections per day. Apomorphine is also extensively metabolized and cannot be administered orally or intravenously. In fact, apomorphine only allows for subcutaneous administration such as via injection or infusion.

The low oral bioavailability of L-dopa and apomorphine is associated with the presence of a catechol moiety in these compounds. In order to reach the bloodstream and enable transport to the brain most of the pharmaceutical drug has to pass through the gastrointestinal tract and the liver, where most catecholamines are subjected to rapid biotransformation. By introducing protecting groups at the hydroxyl and/or the amino functions of the compound, the oral bioavailability can be increased by e.g. slowing down the transformation into the active metabolite and/or allowing the protected drug to function as a prodrug which may release the drug by removal of the protective group by cleavage.

One such a prodrug of dopamine is for instance docarpamine wherein the two hydroxyl groups of dopamine are protected as ethyl carbonate esters and its amino group is protected with an acetyl methionine moiety.

J. Med. Chem 2006, 49, 1494-1498, describes enone prodrugs of dopaminergic catecholamines in the research area of dopamine receptor agonists. It is disclosed that the (−)-enantiomer of the trans-isomer of the compound designated as 1-propyl-2,3,4,4a,5,7,8,9,10,10a-decahydro-1H-benzo[g]quinolin-6-one (also named Compound 4) acts as an enone prodrug of a dopamine receptor agonist. It is suggested that said enone compound is converted in vivo to the corresponding catechol compound designated as N-(n-propyl)-6,7-di-OH-benzo[g]quinoline (also named Compound 3).

Bioorganic & Medicinal Chemistry, 16 (2008), 3438-3444, discloses a synthesis and pharmacological evaluation of a compound titled as racemic trans-1-propyl-1,2,3,4,4a,5,10,10a-octahydrobenzo[g]quinoline-6,7-diol (also named Compound 4), which is believed to be the active form of its enone prodrug. It is stated that the catechol moiety of this compound is decisive for the low bioavailability observed. Further, it is stated that the high efficiency of the compound results in the possibility of administering low dose, which could make it a candidate for the treatment of Parkinson's disease.

WO 2010/097092 describes compounds for treating dyskinesia related disorders, such as Parkinson's disease. The compound (4aR,10aR)-1-propyl-1,2,3,4,4a,5,10,10a-octahydrobenzo[g]quinoline-6,7-diol (also named Compound 10) was found to be an active metabolite functioning as a potent agonist at both the D1 and D2 receptors in vitro and possessing a superior profile as a dopamine agonist in vivo. It is also described that the compound designated as (4aR,10aR)-n-1-propyl-2,3,4,4a,5,7,8,9,10,10a-decahydro-1H-benzo[g]quinolin-6-one (also named Compound 12) may be used for preparing the aforementioned metabolite as well as in the preparation of a medicament for treating Parkinson's disease while maintaining a low dyskinesia induction profile.

WO 2001/078713 discloses maleate salts of the two enantiomers of the compound designated as 1-propyl-trans-2,3,4,4a,5,7,8,9,10,10a-decahydrobenzo[g]quinoline-6-one.

WO 2019/101917 discloses catecholamine prodrugs for use in the treatment in Parkinson's disease. More specifically, it is stated that the invention relates to new prodrug derivatives of the compound (4aR,10aR)-1-n-Propyl-1,2,3,4,4a,5,10,10a-octahydro-benzo[g]quinoline-6,7-diol, and it is reported that glucuronide conjugates and sulfate conjugates of this compound are orally active prodrugs of this compound.

WO 2020/234270 and WO 2020/234271 both disclose processes for the manufacture of the catecholamine prodrug (2S,3S,4S,5R,6S)-3,4,5-trihydroxy-6-(((4aR,10aR)-7-hydroxy-1-propyl-1,2,3,4,4a,5,10,10a-octahydrobenzo[g]quinolin-6-yl)oxy)tetrahydro-2H-pyran-2-carboxylic acid. It is stated that said catecholamine prodrug is for use in the treatment of neurodegenerative diseases and disorders such as Parkinson's disease.

WO 2020/234272 discloses a new solid form of the catecholamine prodrug (2S,3S,4S,5R,6S)-3,4,5-trihydroxy-6-(((4aR,10aR)-7-hydroxy-1-propyl-1,2,3,4,4a,5,10,10a-octahydrobenzo[g]quinolin-6-yl)oxy)tetrahydro-2H-pyran-2-carboxylic acid. It is stated that said catecholamine prodrug is for use in the treatment of neurodegenerative diseases such as Parkinson's disease.

WO 2020/234273 discloses a process for the manufacture of the two compounds (6aR,10aR)-7-propyl-6,6a,7,8,9,10,10a,11-octahydro-[1,3]dioxolo[4′,5′,5,6]benzo[1,2-g]quinoline and (4aR,10aR)-1-propyl-1,2,3,4,4a,5,10,10a-octahydro-benzo[g]quinoline-6,7-diol. It is stated that the compounds are for use in the treatment of neurodegenerative diseases such as Parkinson's disease.

WO 2020/234274, WO 2020/234275, WO 2020/234276, and WO 2020/234277 disclose different prodrugs of the catecholamine (4aR,10aR)-1-propyl-1,2,3,4,4a,5,10,10a-octahydro-benzo[g]quinoline-6,7-diol. The compounds are for use in the treatment of neurodegenerative or neuropsychiatric diseases such as Parkinson's disease.

The listing or discussion of an apparently prior-published document in this specification should not necessarily be taken as an acknowledgement that the document is part of the state of the art or is common general knowledge.

The prior art compound 1-propyl-trans-2,3,4,4a,5,7,8,9,10,10a-decahydro-1H-benzo[g]quinolin-6-one (hereinafter named as (4aR,10aR)-1-propyl-1,2,3,4,4a,5,8,9,10,10a-decahydrobenzo[g]quinolin-6(7H)-one), either as the racemate or as its (4aR,10aR)-enantiomer, is as mentioned above an orally active prodrug of an extremely potent dopamine receptor agonist. However, administration of these compounds is associated with a risk for quickly obtaining high peak plasma concentrations and/or emergent side effects such as nausea and vomiting.

Furthermore, in Parkinson's disease, degeneration of the nigro-striatal dopamine pathways is associated with the core motor symptoms. This deficit is addressed by available dopamine receptor agonists. However, there is also a degeneration of other dopaminergic pathways of the brain. In particular, degeneration of the mesolimbic dopamine pathways is associated with important non-motor symptoms such as depression and apathy in Parkinson's disease.

There is a need for novel therapeutic agents allowing for treatment of CNS diseases, disorders and/or conditions such as Parkinson's disease. In particular, there is a need for a therapeutic agent that is potent, has a long duration of action and/or has few side effects. Further, there is also a need for a therapeutic agent allowing for treating non-motor symptoms associated with Parkinson's disease.

### SUMMARY

It is an object of the present disclosure to provide novel therapeutically active compounds that at least partly overcome or mitigate some of the drawbacks of the aforementioned compounds. A further object is to provide novel therapeutically active compounds useful in the treatment of a CNS disease, disorder and/or condition such as Parkinson's disease. Still a further object of the present disclosure is to provide novel therapeutically active compounds that are potent, have a long duration of action and/or have few side effects, such as nausea and vomiting, when used in the treatment of a CNS disease, disorder and/or condition such as Parkinson's disease. It is also an object of the present disclosure to provide a novel therapeutic agent allowing for treating non-motor symptoms associated with Parkinson's disease. It is also an object of the present disclosure to provide aspects and/or advantages not provided by hitherto known techniques.

The present disclosure provides a compound of Formula III:

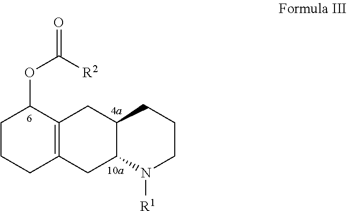

or a pharmaceutically acceptable salt thereof,
wherein
carbon 4a and carbon 10a both have R configuration,
R₁ is methyl, ethyl or n-propyl, and
R₂ is C₁-C₁₀alkyl group.

The present disclosure also provides a pharmaceutical composition comprising a therapeutically acceptable amount of a compound of Formula III as described herein, or a pharmaceutically acceptable salt thereof, in admixture with at least one pharmaceutically acceptable carrier, excipient and/or diluent.

Further, there is also provided a compound of Formula III as described herein, or a pharmaceutically acceptable salt thereof, or
a pharmaceutical composition as described herein
for use as a medicament.

There is also provided a compound of Formula III as described herein, or a pharmaceutically acceptable salt thereof, or
a pharmaceutical composition as described herein
for use in the treatment of one or more of the following:
Parkinson's disease, Huntington's disease, Restless leg syndrome, Alzheimer's disease, schizophrenia, attention deficit hyperactivity disorder, drug addiction.

The present disclosure also provides use of a compound of Formula III as described herein, or a pharmaceutically acceptable salt thereof, or
a pharmaceutical composition as described herein
for the manufacture of a medicament for use in the treatment of one or more of the following: Parkinson's disease, Huntington's disease, Restless leg syndrome, Alzheimer's disease, schizophrenia, attention deficit hyperactivity disorder, drug addiction.

The present disclosure also provides a method for treatment of one or more of the following:
Parkinson's disease, Huntington's disease, Restless leg syndrome, Alzheimer's disease, schizophrenia, attention deficit hyperactivity disorder, drug addiction,
said method comprising administering to a mammal, such as a human or an animal, in need thereof, an effective amount of (i) a compound of Formula III as described herein, or a pharmaceutically acceptable salt thereof, or (ii) a pharmaceutical composition as described herein.

The present disclosure also provides a method for preparing a compound of Formula III, or a pharmaceutically acceptable salt thereof, as described herein, said method comprising the steps of:
a) reacting a compound of Formula II with a compound of Formula IV

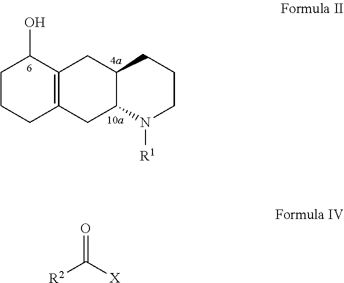

wherein
carbon 4a and carbon 10a in the compound of Formula II both have R configuration, X is selected from the group consisting of OH, halide and OC(O)R², and
R¹ and R² independently are as described herein,
optionally in the presence of an ester forming promoting agent such as a coupling reagent thereby forming the compound of Formula III,
b) optionally separating the compound of Formula III, into a compound of Formula IIIa and a compound of Formula IIIb as defined herein, and
c) optionally combining the compound of Formula III of step a) or step b) with a pharmaceutically acceptable acid thereby providing a pharmaceutically acceptable salt of the compound of Formula III.

### BRIEF DESCRIPTION OF THE DRAWINGS

FIG. 1 shows a reaction scheme for the preparation of a compound of Formula III1.

FIG. 2 shows a chromatogram for the 6S and 6R epimers of (4aR,10aR)-1-propyl-1,2,3,4,4a,5,6,7,8,9,10,10a-dodecahydrobenzo[g]quinolin-6-ol.

FIG. 3 shows effects on locomotor activity for the prior art compound according to Preparation 4A when said compound was administered subcutaneously.

FIG. 4A shows effects on locomotor activity for the compound of the present disclosure according to Example 5 when said compound was administered perorally.

FIG. 4B shows effects on locomotor activity for the compound of the present disclosure according to Example 5 when said compound was administered subcutaneously.

FIG. 5 shows effects on gene expression (mRNA) of Arc in different brain regions by the prior art compound according to Preparation 4A and by the compound according to Example 5.

### DESCRIPTION

The present disclosure provides a compound of Formula III:

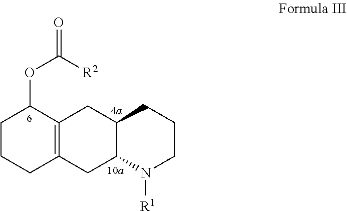

or a pharmaceutically acceptable salt thereof,
wherein
carbon 4a and carbon 10a both have R configuration,
R¹ is methyl, ethyl or n-propyl, and
R² is C₁-C₁₀alkyl.

The term “C₁-C₁₀alkyl” denotes a straight or branched, saturated, acyclic or cyclic alkyl group of one to ten carbon atoms such as methyl, ethyl, n-propyl, iso-propyl, cyclopropyl, n-butyl, isobutyl, sec-butyl, tert-butyl, cyclobutyl, pentyl, cyclopentyl, hexyl, heptyl, octyl, nonyl, decyl. Alternatively, the alkyl group may be unsaturated thereby forming a C₂-C₁₀alkene comprising from two to ten carbons. The C₂-C₁₀alkene may be straight, branched or acyclic and may comprise one or more double bonds. Examples of C₂-C₁₀alkene include vinyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl, 1-heptenyl, 1-octenyl, 1-nonenyl. The cyclic alkyl group, i.e. a cycloalkyl, may comprise from three to ten carbon atoms and may comprise one or more cyclic groups. Examples of the cyclic alkyl group include cyclopropyl, cyclobutyl, cyclopentyl, cyclohexyl, cycloheptyl, cyclooctyl, cyclononyl and cyclodecyl. Further, the cyclic alkyl group may be part-cyclic such as for example cyclopropylmethyl. Moreover, the cyclic alkyl group may comprise one or more double bonds.

As used herein, the term “halide” may be Cl, Br or I.

Thus, R¹ of the compounds described herein may be methyl, ethyl or n-propyl. For instance, R¹ may be ethyl or n-propyl. In an example, R¹ is ethyl. In a further example R¹ is n-propyl.

The R² substituent is C₁-C₁₀alkyl. For instance, the R² substituent may be methyl, ethyl, propyl or butyl. In an example, R² is methyl.

The compound of Formula III may exist as a compound of Formula IIIa and/or a compound of Formula IIIb:

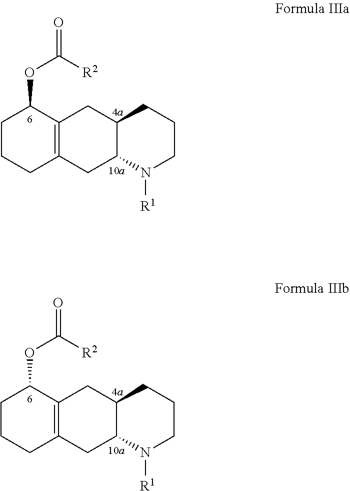

It will be appreciated that R¹ and R² for the compounds of Formula IIIa and Formula IIIb, respectively, may be as described herein. Further, it is understood that the compounds of Formula IIIa and Formula IIIb are epimers that differ in configuration on carbon 6 (i.e. the carbon linked to the OC(O)R² group). The compound of Formula IIIa is the 6R epimer while the compound of Formula IIIb is the 6S epimer. For the compounds of Formula IIIa and Formula IIIb the stereochemistry of the tricyclic system is as depicted herein, i.e. the ring containing the nitrogen exhibits trans configuration wherein the carbon 4a has R configuration and the carbon 10a has R configuration. The numbering of the carbon atoms for the compounds of Formula III, Formula IIIa and Formula IIIb is shown below.

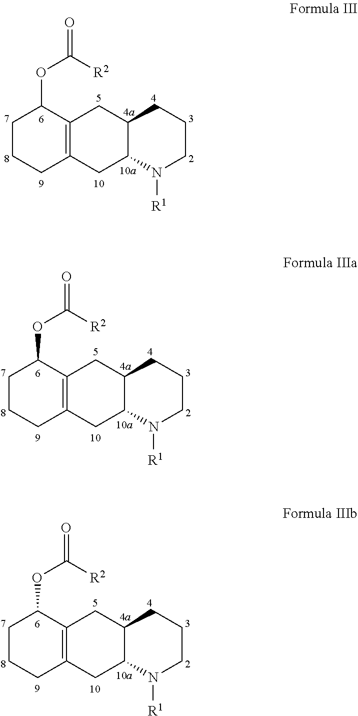

In an example, the compound of the present disclosure is a compound of Formula IIIa. In a further example, the compound of the present disclosure is a compound of Formula IIIb. In still a further example, the compound of Formula III is a mixture of the compound of Formula IIIa and the compound of the compound of Formula IIIb, such as a 1:1 mixture of the compound of Formula IIIa and the compound of Formula IIIb.

It will be appreciated that the nitrogen atom of the compounds disclosed herein may be provided in oxidized form such as a compound of Formula III2. Persons skilled in the art will understand that such compounds may be administered to a patient or formed in vivo after administration to a patient.

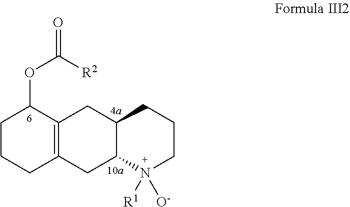

The present disclosure also provides a compound of Formula III1 wherein R¹ is n-propyl and R² is methyl. The compound of Formula III1 may be denominated (4aR,10aR)-1-propyl-1,2,3,4,4a,5,6,7,8,9,10,10a-dodecahydrobenzo[g]quinolin-6-yl acetate.

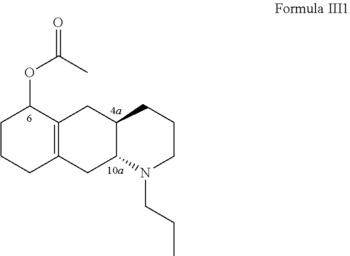 [SMILES: CCCN1CCC[C@@H]2CC3=C(CCCC3OC(C)=O)C[C@H]21]

It will be appreciated that the compound of Formula III1 is an isomeric mixture of the 6R and 6S epimers, i.e. an isomeric mixture of the compound of Formula IIIa1 and the compound of Formula IIIb1 described herein.

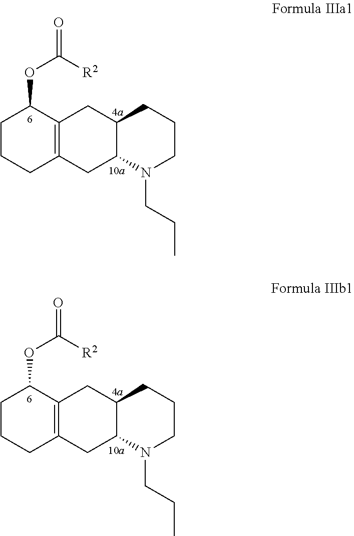

It will be appreciated that the compound of Formula IIIa1 is the 6R epimer of the compound of Formula III1 and may be denominated (4aR,6R,10aR)-1-propyl-1,2,3,4,4a,5,6,7,8,9,10,10a-dodecahydrobenzo[g]quinolin-6-yl acetate. Further, it will be appreciated that the compound of Formula IIIb1 is the 6S epimer of the compound of Formula III1 and may be denominated (4aR,6S,10aR)-1-propyl-1,2,3,4,4a,5,6,7,8,9,10,10a-dodecahydrobenzo[g]quinolin-6-yl acetate.

The compounds described herein may be provided as a single diastereomer such as a diastereomer essentially free of any other diastereomer. The single diastereomer may have R configuration at carbon 6, and have R configuration both at carbon 4a and at carbon 10a. Alternatively, the single diastereomer may have S configuration at carbon 6, and have R configuration both at carbon 4a and at carbon 10a. Further, the single diastereomer may be provided in a diastereomeric excess equal to or above 90%, such as equal to or above 95%, or such as equal to or above 99%. As used herein, the diastereomeric excess equals percentage of the major diastereomer minus percentage of the minor diastereomer. For instance, a mixture composed of 95% of the major diastereomer and 5% of the minor diastereomer has a diastereomeric excess of 90%.

The present disclosure provides a pharmaceutically acceptable salt of the compound(s) described herein, such as the compounds of Formula III, Formula IIIa, Formula IIIb, Formula III2, Formula III1, Formula IIIa1 and Formula IIIb1.

The pharmaceutically acceptable salt of the compounds described herein may be provided as a combination of a compound of Formula III as described herein and an organic acid. Further, the pharmaceutically acceptable salt of the compounds described herein may be provided as a combination of a compound of Formula III as described herein and an organic acid in a ratio of 1:1 or 2:1. In an example, the ratio is 1:1. In a further example, the ratio is 2:1. The organic acid may be D-tartaric acid.

For instance, there is provided a salt of Formula III11

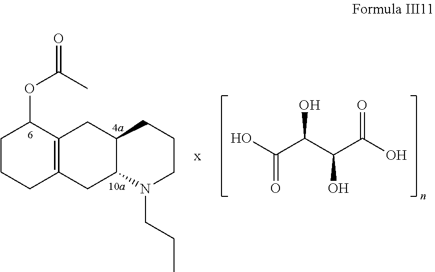

said salt being a combination of the compound of Formula III1 and D-tartaric acid:

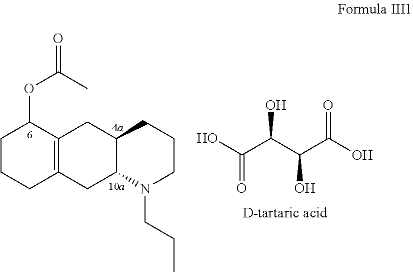

in a ratio of 1:n,
wherein
n is 0.5 or 1. In particular, n may be 1.

Further, there is provided a salt of Formula IIa11

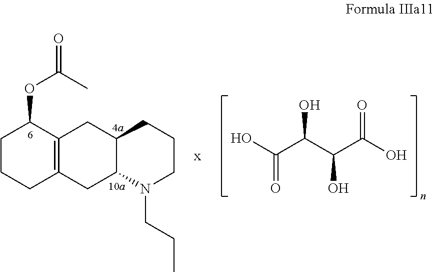

said salt being a combination of the compound of Formula IIIa1 and D-tartaric acid:

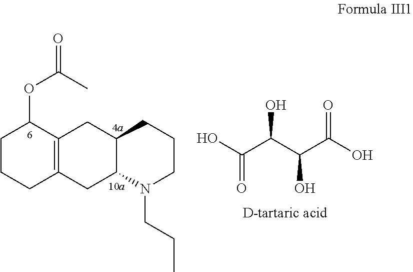

in a ratio of 1:n,
wherein
n is 0.5 or 1. In particular, n may be 1.

There is also provided a salt of Formula IIIb11

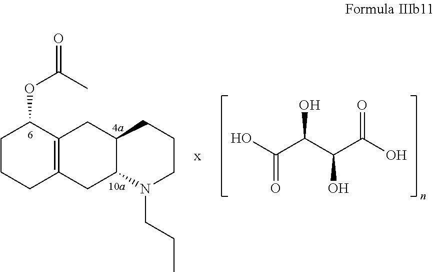

said salt being a combination of the compound of Formula IIIb1 and D-tartaric acid:

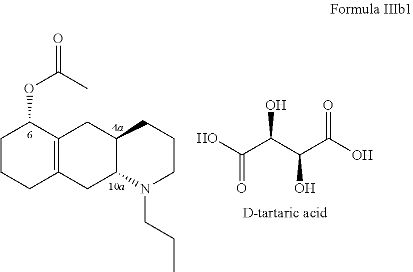

in a ratio of 1:n,
wherein
n is 0.5 or 1. In particular, n may be 1.

The compounds described herein, or a pharmaceutically acceptable salt thereof, may exist in solid form, i.e. they may be provided as a solid. For example, the compounds described herein, or a pharmaceutically acceptable salt thereof, may be amorphous, crystalline or a mixture thereof. Further, the compounds described herein, or a pharmaceutically acceptable salt thereof, may exist in crystalline form, i.e. they may be provided as crystal(s). The degree of crystallinity may be equal to or above 80%, 85%, 90%, 95% or 99%.

The compound of Formula III described herein, or a pharmaceutically acceptable salt thereof, may be included in a pharmaceutical composition. Thus, there is provided a pharmaceutical composition comprising a therapeutically acceptable amount of a compound of Formula III as described herein, or a pharmaceutically acceptable salt thereof, in admixture with at least one pharmaceutically acceptable carrier, excipient and/or diluent. As used herein, the expression “therapeutically effective amount” means an amount of a compound as disclosed herein that is sufficient to induce the desired therapeutic effect in a patient to which the compound is administered. The pharmaceutical composition may be for oral administration. Additionally or alternatively, the pharmaceutical composition may be for rectal, intracisternal, intravaginal, intraperitoneal and/or parenteral administration. In an example, the parenteral administration may be intravenous, intramuscular or subcutaneous administration. Further, the pharmaceutical composition may be provided in solid form such as in the form of one or more capsules, tablets, pills, powders and/or granules. Alternatively, the pharmaceutical composition may be provided in liquid form such as in the form of one or more emulsions, solutions, suspensions and/or syrups.

There is also provided a compound of Formula III as described herein, or a pharmaceutically acceptable salt thereof, or
a pharmaceutical composition as described herein
for use as a medicament.

There is also provided a compound of Formula III as described herein, or a pharmaceutically acceptable salt thereof,
or a pharmaceutical composition as described herein
for use in the treatment of one or more of the following:
Parkinson's disease, Huntington's disease, Restless leg syndrome, Alzheimer's disease, schizophrenia, attention deficit hyperactivity disorder, drug addiction. For instance, the treatment and may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

As used herein, the term treatment may involve one or more of the following: therapeutic treatment, palliative treatment, treatment reducing worsening or the development of a disorder or disease as described herein. For instance, the treatment may be therapeutic treatment and/or palliative treatment. Additionally or alternatively, the treatment may be treatment reducing worsening or the development of a disorder or disease as described herein.

The present disclosure also provides use of a compound of Formula III as described herein, or a pharmaceutically acceptable salt thereof,
or a pharmaceutical composition as described herein
for the manufacture of a medicament for use in the treatment of one or more of the following: Parkinson's disease, Huntington's disease, Restless leg syndrome, Alzheimer's disease, schizophrenia, attention deficit hyperactivity disorder, drug addiction. For instance, the treatment may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

The present disclosure also provides a method for treatment of one or more of the following:
Parkinson's disease, Huntington's disease, Restless leg syndrome, Alzheimer's disease, schizophrenia, attention deficit hyperactivity disorder, drug addiction,
said method comprising administering to a mammal, such as a human or an animal, in need thereof, an effective amount of (i) a compound of Formula III as described herein, or a pharmaceutically acceptable salt thereof, or (ii) a pharmaceutical composition described herein. For instance, the disease, condition and/or disorder may involve Parkinson's disease. Thus, there is provided a method for treatment of Parkinson's disease comprising administering to a mammal, such as a human or an animal, in need thereof, an effective amount of (i) a compound of Formula III as described herein, or a pharmaceutically acceptable salt thereof, or (ii) a pharmaceutical composition as described herein. For instance, the treatment may comprise or consist of treatment of Parkinson's disease. In a further example, the treatment may comprise or consist of treatment of Huntington's disease or Restless leg syndrome. In still a further example, the treatment may comprise or consist of treatment of Alzheimer's disease or schizophrenia. In yet an example, the treatment may comprise or consist of treatment of attention deficit hyperactivity disorder (ADHD) or drug addiction.

As used herein, Parkinson's disease includes motor symptoms with or without non-motor symptoms. The main motor symptoms include tremor, rigidity, slowness of movement and difficulty in walking. Collectively, these main motor symptoms are known as “parkinsonism” or “parkinsonian syndrome”. Non-motor symptoms include cognitive functional decline, depression, anxiety, apathy and dementia.

The treatment of Parkinson's disease described herein may take place without or substantially without inducing side effects such as nausea or vomiting. Thus, the treatment may be associated with no or mild side effects such as nausea and/or vomiting.

Additionally or alternatively, the treatment described herein may comprise or consist of non-motor symptoms associated with Parkinson's disease. Examples of non-motor symptoms include cognitive functional decline, depression, anxiety, apathy and/or Parkinson's disease dementia. Examples of cognitive functional decline include problems with memory, language, thinking, learning and/or judgment.

The present disclosure also provides a method for preparing a compound of Formula III as described herein, or a pharmaceutically acceptable salt thereof, said method comprising the steps of:
a) reacting a compound of Formula II with a compound of Formula IV

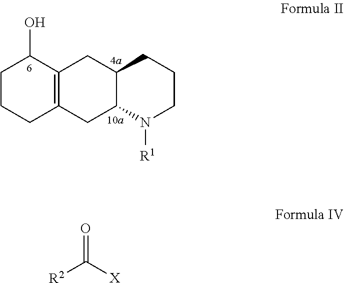

wherein
carbon 4a and carbon 10a in the compound of Formula II have R configuration,
X is selected from the group consisting of OH, halide and OC(O)R², and
R¹ and R² independently are as described herein,
optionally in the presence of an ester forming promoting agent such as a coupling reagent thereby forming the compound of Formula III,
b) optionally separating the compound of Formula III, into a compound of Formula IIIa and a compound of Formula IIIb as defined in any one of claims 2-10, and
c) optionally combining the compound of Formula III of step a) or step b) with a pharmaceutically acceptable acid thereby providing a pharmaceutically acceptable salt of the compound of Formula III.

The pharmaceutically acceptable acid in step c) may be a pharmaceutically acceptable acid as described herein. In particular, the pharmaceutically acceptable acid may comprise or consist of D-tartaric acid.

As used herein, the ester forming promoting agent may be a coupling agent such as dicyclohexylcarbodiimid (DCC) or diisopropylcarbodiimid (DIC).

It will be appreciated that the compound of Formula IV as described herein may be an anhydride such as acetic acid anhydride.

The compound of Formula II used in the method for preparing a compound of Formula III, or pharmaceutically acceptable salt thereof, as described herein may comprise a compound of Formula IIa and/or Formula IIb. In an example, the compound of Formula II is provided as a mixture of the compound of Formula IIa and the compound of Formula IIb such as a 1:1 mixture. Alternatively, the compound of Formula II may be provided as a compound of Formula IIa or a compound of Formula IIb.

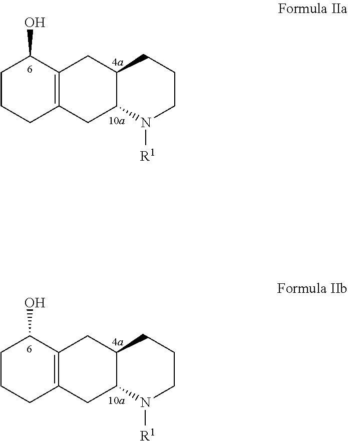

It will be appreciated that R¹ for the compounds of Formula IIa and Formula IIb may be as described herein. Further, it is understood that the compounds of Formula IIa and Formula IIb are epimers that differ in configuration on carbon 6 (i.e. the carbon linked to the OH group). The compound of Formula IIa is the 6R epimer while the compound of Formula IIb is the 6S epimer.

The R¹ group of the compounds of Formula II, Formula IIa and Formula IIb may be n-propyl thereby providing a compound of Formula II1, Formula IIa1 and/or Formula IIb1.

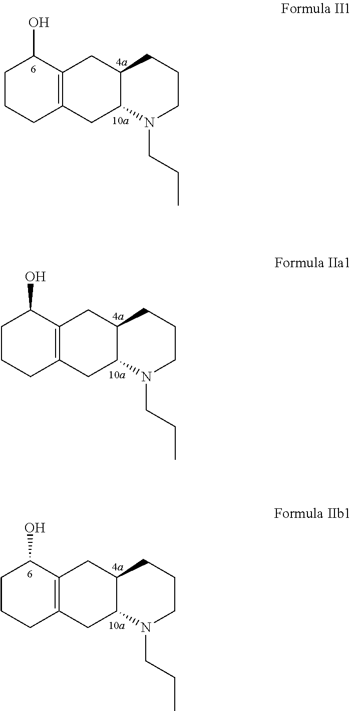 [SMILES: CCCN1CCC[C@@H]2CC3=C(CCCC3O)C[C@H]21 ; CCCN1CCC[C@@H]2CC3=C(CCC[C@H]3O)C[C@H]21 ; CCCN1CCC[C@@H]2CC3=C(CCC[C@@H]3O)C[C@H]21]

The chemical name of the compound of Formula II1 may be (4aR,10aR)-1-propyl-1,2,3,4,4a,5,6,7,8,9,10,10a-dodecahydrobenzo[g]quinolin-6-ol.

The chemical name of the compound of Formula IIa1 may be: (4aR,6R,10aR)-1-propyl-1,2,3,4,4a,5,6,7,8,9,10,10a-dodecahydrobenzo[g]quinolin-6-ol.

The chemical name of the compound of Formula IIb1 may be: (4aR,6S,10aR)-1-propyl-1,2,3,4,4a,5,6,7,8,9,10,10a-dodecahydrobenzo[g]quinolin-6-ol.

The compound of Formula II described herein may be prepared by a process comprising the steps of:
a) reducing a compound of Formula I

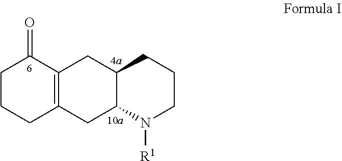

wherein R¹ is as described herein,
with a reducing agent in the presence of a Lewis acid and a solvent
whereby the carbonyl group is reduced into a hydroxyl group thereby providing the compound of Formula II, and
b) optionally separating the compound of Formula II into a compound of Formula IIa and Formula IIb.

In the method for preparing the compound of Formula II the reducing agent, the Lewis acid and the solvent are selected so as to reduce the carbonyl selectively into a hydroxyl group, i.e. the carbonyl group is reduced into a hydroxyl group without or substantially without reducing the double bond into a single bond. The reducing agent may be a hydride such as sodium borohydride, and/or the Lewis acid may comprise cerium(III) chloride such as cerium(III) chloride heptahydrate, and/or the solvent may comprise a protic solvent such as an alcohol such as methanol. This will generally provide a 1:1 mixture of compounds of Formula IIa and Formula IIb. A chiral reducing agent may be used if it is desired to produce an excess of the compound of Formula IIa or the compound of Formula IIb. For example, a chiral alkylborohydride such as diisopinocampheylborane may be used. Methods commonly known in the art may be used in order to obtain the compound of Formula IIa or the compound of Formula IIb in chemically and/or stereochemically pure form.

It will be appreciated that the R¹ group of the compound of Formula I may be n-propyl thereby providing a compound of Formula I1, which may have the chemical name (4aR,10aR)-1-propyl-1,2,3,4,4a,5,8,9,10,10a-decahydrobenzo[g]quinolin-6(7H)-one.

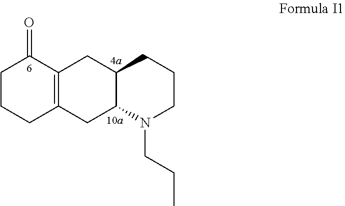 [SMILES: CCCN1CCC[C@@H]2CC3=C(CCCC3=O)C[C@H]21]

### Pharmaceutically Acceptable Salts

Compounds of the present disclosure may be provided in the form of a pharmaceutically acceptable salt. As used herein “pharmaceutically acceptable salt”, where such salt is possible, includes salt prepared from a pharmaceutically acceptable non-toxic acid, i.e. pharmaceutically acceptable acid addition salts. The pharmaceutically acceptable salt may be formed by combining a compound as described herein with an organic acid or inorganic acid in a desired ratio using e.g. methods known in the art. Thus, the pharmaceutically acceptable salt may be a combination of the compound of Formula III and an acid such as a combination of the compound of Formula III and an acid in a ratio of 1:1 or 2:1.

Examples of pharmaceutically acceptable salts include, without limitation, non-toxic inorganic and organic acid addition salts such as hydrochloride, hydrobromide, borate, nitrate, perchlorate, phosphate, sulphate, formate, acetate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, propionate, salicylate, sorbate, stearate, succinate, tartrate, toluene-p-sulphonate, and the like.

Other acids such as oxalic acid, may be useful in the preparation of salts useful as intermediates in obtaining a compound of the present disclosure and its pharmaceutically acceptable acid addition salt.

### Solvates

It is to be understood that compounds of the present disclosure, a pharmaceutically acceptable salt thereof, may exist in solvated form. Alternatively, the compounds of the present disclosure, or a pharmaceutically acceptable salt thereof, may exist in non-solvated forms. The term “solvate” is used herein to describe a molecular complex comprising a compound of the present disclosure and one or more pharmaceutically acceptable solvent molecule(s). The term “hydrate” is employed when the solvent is water. Thus, solvated forms may include hydrated forms such as monohydrate, dihydrate, hemihydrate, trihydrate, tetrahydrate, and the like.

### Polymorphs

The compounds of the present disclosure, or a pharmaceutically acceptable salt thereof, may exist in a continuum of solid states ranging from fully amorphous to fully crystalline. Thus, it is to be understood that the compounds of the present disclosure may be in the form of a polymorph.

### Labelled Compounds

The compounds of the present disclosure, or a pharmaceutically acceptable salt thereof, may be used in their labelled or unlabelled form. In the context of this present disclosure the labelled compound has one or more atoms replaced by an atom having an atomic mass or mass number different from the atomic mass or mass number usually found in nature. The labelling may allow easy quantitative detection of said compound.

For example, there is provided a compound as described herein which is labelled with one or more isotopes, such as for example tritium (³H), deuterium (²H), or carbon-14 (¹⁴C). In an example, the compound is labelled with one or more deuterium atoms. All isotopic variations of the compounds of the present disclosure, whether radioactive or not, are intended to be encompassed within the scope of the present disclosure.

Thus, the present disclosure provides a compound as described herein which is labelled with one or more isotopes such as deuterium. The compounds labelled with an isotope as described herein may be combined with an acid as described herein thereby providing a salt such as a pharmaceutically acceptable salt as described herein.

### Dosage

It will be appreciated that the compounds described herein may be administered in a therapeutically acceptable amount. For example, the dose may be from about 0.0001 mg/kg bodyweight to about 5 mg/kg bodyweight, such as from 0.001 mg/kg bodyweight to about 1 mg/kg bodyweight. The exact dosages will depend upon the frequency and mode of administration, the sex, the age, the weight, and the general condition of the subject to be treated, the nature and the severity of the condition to be treated, any concomitant diseases to be treated, the desired effect of the treatment and/or other factors known to those skilled in the art.

### Methods of Preparation

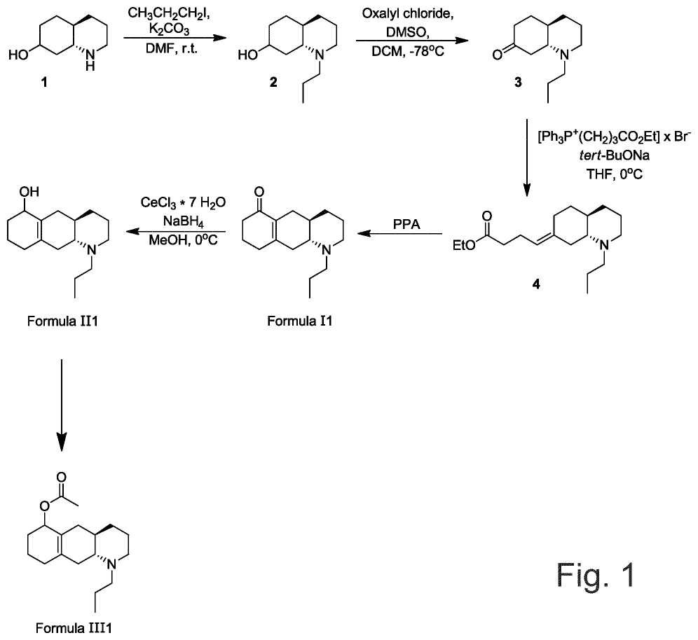

Compounds of the present disclosure may be prepared as described herein. For instance, the compounds of the present disclosure may be prepared as shown in FIG. 1.

The end products of the reactions described herein may be isolated by conventional techniques, e.g. by extraction, crystallization, distillation, chromatography, etc. The compounds of the present disclosure may be prepared in chemically pure form, i.e. they are substantially free from reactants, solvents, impurities etc. Further, the compounds of the present disclosure may be prepared in substantially stereochemically pure form. For instance, the compound of Formula III may contain the compound of Formula IIIa and the compound of Formula IIIb in a ratio equal to or above 95:5, 96:4, 97:3, 98:2 or 99:1. In a further example, the compound of Formula III may contain the compound of Formula IIIb and the compound of Formula IIIa in a ratio equal to or above 95:5, 96:4, 97:3, 98:2 or 99:1.

Persons skilled in the art will appreciate that, in order to obtain compounds of the present disclosure the individual process steps mentioned hereinbefore may be performed in a different order, and/or the individual reactions may be performed at different stage in the overall route (i.e. chemical transformations may be performed upon different intermediates to those associated hereinbefore with a particular reaction).

The disclosure is illustrated in the following non-limitative Examples.

### EXAMPLES

The naming of the compounds (preferred IUPAC name) as disclosed herein was made using ChemDraw Ultra, version 12.0.2. 1076. In this document, if the chemical name and the chemical structure are inconsistent the chemical structure should be considered to be the correct structure.

### General Methods

Chemicals were mainly purchased from Sigma-Aldrich. Preparative HPLC was performed on a Gilson system equipped with a UV detector. For flash chromatography, Biotage Isolera Vers 1.2 with Sfar Silica HC D columns were used. Analytical HPLC/LCMS was performed using an Agilent 1100 series Liquid Chromatograph/Mass Selective Detector (MSD) (Single Quadropole) equipped with an electrospray interface and a UV diode array detector. Analyses were performed by using either an ACE 3 C8 (3.0×50 mm) column with a 10-97% gradient of acetonitrile in 0.1% aqueous TFA over 3 min and a flow of 1 mL/min, or an Xbridge C18 (3.0×50 mm) column with a 10-97% gradient of acetonitrile in 10 mM ammonium bicarbonate over 3 min and a flow of 1 mL/min and UV detection. Alternatively, a 10-100% gradient of acetonitrile in 0.03% acetic acid was used as eluent. Low resolution mass spectra were recorded on a HP 5970A instrument operating at an ionization potential of 70 eV. The mass detector was interfaced with a HP5700 gas chromatograph equipped with a HP-5MS UI GC column (15m, 0.25 mm, 0.25 μm) with a helium gas flow of 40 cm/s.

Optical measurements were performed at 25° C. using a sodium lamp (A=589 nM). ¹H-NMR spectra were recorded on a Varian 400 MHz instrument at 25° C. or on a Bruker 600, 700, 800 or 900 MHz instrument when specified. ¹³C NMR spectrum was obtained using a Bruker instrument operating at 200 MHz.

Melting points were determined by a Buchi B-545 apparatus and are uncorrected.

### Abbreviations

AP anterior posterior
aq. Aqueous
ARC activity-regulated cytoskeleton-related protein
Ctrl Control
d doublet
dd doublet of doublets
DCM dichloromethane
DEA diethylamine
DMF dimethylformamide
DMSO dimethsulphoxide
egr-1 early growth response protein 1
EtOAc ethyl acetate
EtOH ethanol
g gram(s)
mg milligram(s)
kg kilogram(s)
i.p. intraperitoneal
IP intraperitoneal
IUPAC International Union of Pure and Applied Chemistry
HPLC High Performance Liquid Chromatography
HPRT Hypoxanthine-guanine phosphoribosyltransferase
M molar, i.e. mole(s)/liter
MeOH methanol
mM millimolar, i.e. millimole(s)/liter
ML medial lateral
MS(ESI+) Mass Spectroscopy Electrospray Ionization
min. minute(s)
mg milligram(s)
mL millilitre(s)
ml millilitre(s)
mol mole
mmol(e) millimole
MS Mass Spectrometry
NMR Nuclear Magnetic Resonance
Npas4 Neuronal PAS Domain Protein 4
PCR Polymerase Chain Reaction
PPA polyphosphoric acid
PO perorally
Rf retention factor
RT Reverse transcriptase
SEM standard error of the mean
SC subcutaneously
td triplet of doublets
THF Tetrahydrofurane
TLC thin layer chromatography
UV ultraviolet
V ventral
Å Angstrom

### Example 1

### Synthesis of (4aR,10aR)-1-propyl-1,2,3,4,4a,5,8,9,10,10a-decahydrobenzo[g]quinolin-6(7H)-one (synthetic intermediate as well as prior art compound; compound of Formula I1)

### A) trans-1-propyldecahydroquinolin-7-ol (Compound 2 in the Synthesis of FIG. 1)

To a stirred solution of trans-decahydroquinolin-7-ol (1.32 mL, 12.9 mmol) in DMF (20 mL) was added 1-iodopropane (2.3 g, 13.5 mmol). After 10 min, potassium carbonate (8.9 g, 64 mmol) was added. The reaction mixture was stirred overnight and was then diluted with 80 mL of water and extracted with 3×30 ml of DCM. The organic layers were combined, washed with brine (4×40 mL) and concentrated to give trans-1-propyldecahydroquinolin-7-ol as a yellow oil (2.5 g, 12.7 mmol, 98% yield).

### B) trans-1-propyloctahydroquinolin-7(1H)-one (Compound 3 in the Synthesis of FIG. 1)

To a stirred solution of oxalyl chloride (2.07 ml, 24 mmol) in DCM (40 mL) at −78° C. was added DMSO (3.5 mL, 49 mmol) dropwise and the reaction mixture was stirred for 15 min at this temperature. Then, a solution of trans-1-propyldecahydroquinolin-7-ol (2.5 g, 12.7 mmol) in 10 mL of DCM was added at −78° C. and the reaction mixture was stirred for 1 h. Triethylamine (14 mL, 101 mmol) was added and the reaction mixture was slowly warmed to room temperature and stirred for 2 h. After this, the reaction mixture was washed with saturated NaHCO₃, water and brine. The organic phase was dried over Na₂SO₄ and concentrated to give trans-1-propyloctahydroquinolin-7(1H)-one as a yellow oil (1.63 g, 8.3 mmol, 66% yield).

### C) (E)-ethyl 4-(trans-1-propyloctahydroquinolin-7(1H)-ylidene)butanoate (Compound 4 in the Synthesis of FIG. 1)

To a stirred suspension of (4-ethoxy-4-oxobutyl) triphenylphosphonium bromide (9.93 g, 22 mmol) in THF (50 mL) at 0° C. was added sodium tert-butoxide (2M in THF, 12.5 mL, mmol) and the reaction mixture was stirred for 30 min. Then, trans-1-propyloctahydroquinolin-7(1H)-one (2.12 g, 10.9 mmol) in THF (10 mL) was added dropwise at 0° C. during 3 min and the reaction mixture was stirred over two days (until starting material disappeared). The reaction mixture was diluted with cold water (50 mL) with ice bath cooling and extracted with hexane (4×50 mL). The solvent was removed in vacuo, and the residue was triturated with 50 mL of hexane whereby a precipitate of Ph₃PO was formed. The precipitate was removed by filtration and the solution was concentrated to give 3.18 g of crude product (purity approximately 50%). The material was used in the next step without additional purification.

### D) (4aR,10aR)-1-propyl-1,2,3,4,4a,5,8,9,10,10a-decahydrobenzo[g]quinolin-6(7H)-one, Synthetic Intermediate in the Preparation of the Compound of Formula III1, which Also May be Prepared According to the Method Disclosed in Reference 1

A solution of (E)-ethyl 4-(trans-1-propyloctahydroquinolin-7(1H)-ylidene)butanoate (3.18 g, 5.42 mmol) in DCM (3 mL) was added dropwise to PPA (30 g) at 100° C. during 5 min (temperature should not be higher than 110° C.) and followed by stirring for 2 h under heating. Then, ice and 50 mL of water were added and the mixture was extracted with DCM to remove impurities. The aqueous phase was basified with NH₃ (28% aq.) and extracted with DCM to give a crude mixture of the two isomers. This was purified by column chromatography (silica, gradient of 1 to 9% MeOH in DCM, with the MeOH containing 1% NH₃ to give the title compound (860 mg, 30% yield over 2 steps). MS (ESI+) m/z 248 [M+H]+. This mixture of enantiomers was separated using a preparative HPLC system equipped with a semipreparative chiral column (CHIRALPAK ID, 5 μm, 10×250 mm) and a mobile phase of heptane/isopropanol/diethylamine 80/20/0.1 with a flow rate of 4.6 mL/min.

### Peak 1:

(4aR,10aR)-1-propyl-1,2,3,4,4a,5,8,9,10,10a-decahydrobenzo[g]quinolin-6(7H)-one (360 mg, 1.45 mmol). Optical measurement: [α]_{D}²⁵=−234° (c=0.035, methanol).

### Peak 2:

(4aS,10aS)-1-propyl-1,2,3,4,4a,5,8,9,10,10a-decahydrobenzo[g]quinolin-6(7H)-one (400 mg, 1.62 mmol). Optical measurement: [α]_{D}²⁵=+228° (c=0.032, methanol).

### Example 2

### Synthesis of (4aR,10aR)-1-propyl-1,2,3,4,4a,5,6,7,8,9,10,10a-dodecahydrobenzo[g]quinolin-6-ol (Mixture of 6R and 6S Epimers, i.e. Compound of Formula II1 in the Synthesis of FIG. 1, which is a Synthetic Intermediate in the Preparation of the Compound of Formula III1)

Cerium(III) chloride heptahydrate (547 mg, 1.47 mmol) was added to a stirred and cooled (0° C.) solution of (4aR,10aR)-1-propyl-1,2,3,4,4a,5,8,9,10,10a-decahydrobenzo[g]quinolin-6(7H)-one (300 mg, 1.21 mmol) in MeOH (8 mL) and stirred 15 min. Sodium borohydride (138 mg, 3.6 mmol) was added in 3 portions over 15 min, and after 1 h the reaction was completed. Water (10 mL) was added to the resulting white slurry and stirred 20 min. The clear aqueous solution was extracted with EtOAc (4×30 mL) and concentrated to give 190 mg of product as a mixture of diastereomers.

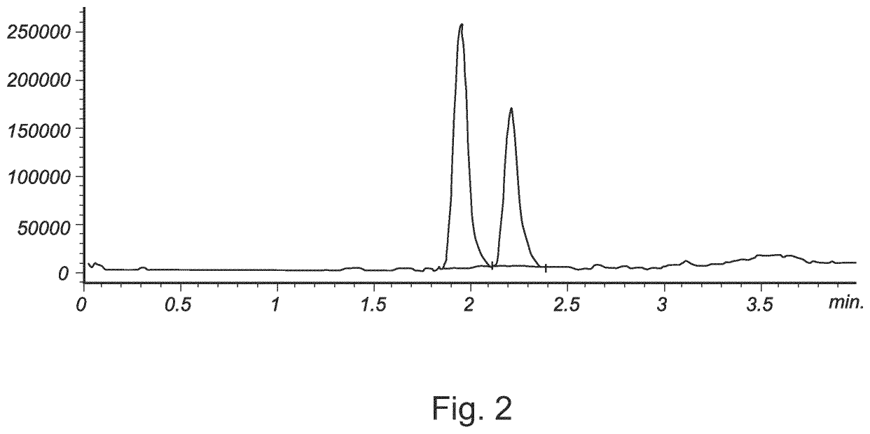

The two diastereomers (epimer 1 and epimer 2) were first separated in analytical scale, which is shown in the chromatogram of FIG. 2 where epimer 1 has the retention time 1.95 min and epimer 2 has 2.21 min under the following conditions: (Xbridge C18, 50×3.0, 3.5 u, 10 to 97% acetonitrile in 10 mM NH₄HCO₃ (pH 10) over 3 min, 1 ml/min.).

The mixture of diastereomers were also, in another experiment, separated using preparative HPLC (XBridge™ Prep C18 5 p.m OBD™ 19×50 mm column, 15 to 40% acetonitrile in 50 mM aqueous NH₄HCO₃ over 6 min). The two epimers were eluted in the same order as in the analytical experiment. One of these is the 6S epimer and the other is the 6R epimer, but at first it was not clear which epimer that elutes first. An X ray analysis then showed that the epimer that elutes first has 6R configuration and the epimer that elutes last has 6S configuration. The yield of Epimer 1 was 110 mg, 36.4% yield, and the yield of epimer 2 was 35 mg, 11.6% yield.

Final evaporation and drying gave two products as pale yellow oils which solidified over time. The isomers were numbered by the order of elution from the column.

### Example 3

### NMR Spectra of the Compounds of Example 2.

### Mixture of Epimer 1 and Epimer 2:

1H NMR (400 MHz, CDCl₃) δ 4.01 (br s, 0.7H), 3.85 (br s, 0.3H), 2.94 (br d, J=11.2 Hz, 1H), 2.56-2.72 (m, 1H), 2.08-2.42 (multiplets), 0.93-1.09 (m, 1H), 0.85 (t, J=7.4 Hz, 3H). The total yield was 48%.

### Epimer 1 (6R Epimer):

1H NMR (400 MHz, CDCl₃): δ 4.02 (br s, 1H), 2.95 (br d, J=11.4 Hz, 1H), 2.59-2.70 (m, 1H), 2.30-2.41 (m, 1H), 2.12-2.29 (m, 2H), 1.28-2.09 (multiplets), 0.96-1.08 (m, 1H), 0.86 (t, J=7.3 Hz, 3H). MS (ESI+) m/z 250 [M+H]+.

### Epimer 2: 1 (6S Epimer):

1H NMR (400 MHz, CDCl₃): δ 3.86 (br s, 1H), 2.95 (br d, J=10.2 Hz, 1H), 2.60-2.72 (m, 1H), 2.30-2.43 (m, 2H), 2.12-2.27 (m, 2H), 1.39-2.04 (multiplets), 0.93-1.08 (m, 1H), 0.86 (t, 1=7.4 Hz, 3H). MS (ESI+) m/z 250 [M+H)+.

### Example 4A

### Preparation 4A (4aR,10aR)-1-propyl-1,2,3,4,4a,5,8,9,10,10a-decahydrobenzo[g]quinolin-6(7H)-one (synthetic intermediate as well as prior art compound; compound of Formula I1)

(E)-ethyl 4-(trans-1-propyloctahydroquinolin-7(1H)-ylidene)butanoate (4.0 g, 13.7 mmol), which had been obtained in a similar fashion as in Example 1 C, was mixed with Eaton's reagent (phosphorus pentoxide, 7.7 wt. % in methanesulfonic acid, 21.1 g) and the mixture was heated at 80° C. for 3 h. The reaction mixture was carefully added dropwise to an ice-cooled aqueous solution of NaHCO₃ (10%). After extracting three times with DCM, the organic solution was dried over Na₂SO₄, filtered and evaporated. The residue was purified by silica-gel chromatography using isooctane/EtOAc/MeOH (gradient, 0-100% EtOAc and then 0-100% MeOH) as eluent. There was obtained 1.4 g of the racemic intermediate and the two enantiomers were thereafter separated on a chiral column (Chiralpak IG, 10 mm×250 mm) using heptane, EtOH and DEA (90:10:0.1) as eluent. Approximately 25 mg of the racemate was each time loaded on the column and the isomer that eluted last from the column was collected. After pooling the desired fractions, the product was again purified by silica-gel chromatography using isooctane/EtOAc/MeOH as eluent (gradient, 0-100% EtOAc and then 0-100% MeOH). There was obtained 0.35 g of (4aR,10aR)-1-propyl-1,2,3,4,4a,5,8,9,10,10a-decahydrobenzo[g]quinolin-6(7H)-one in its non-salt form as an oil. [α]²⁰_{D}=−215.6 (c, 0.010 g/mL, MeOH). ¹H NMR (800 MHz, CDCl₃): b 0.88 (t, 3H), 1.04 (m, 1H), 1.38 (m, 1H), 1.49 (m, 2H), 1.62 (m, 1H), 1.68 (m, 2H), 1.84 (m, 1H), 1.98 (m, 3H), 2.16 (m, 2H), 2.26 (m, 2H), 2.35 (m, 2H), 2.42 (m, 1H), 2.52 (m, 2H), 2.67 (m, 1H), 2.97 (m, 1H). The HCl-salt of (4aR,10aR)-1-propyl-1,2,3,4,4a,5,8,9,10,10a-decahydrobenzo[g]quinolin-6(7H)-one was prepared by mixing 518 mg (2.1 mmol) of its non-salt form (synthesized in a similar fashion as above) with HCl in ethanol (1.25 M, 4 mL) and then concentrating the formed solution on a rotavapor. The residue was co-evaporated with ethanol and then crystallized from ethanol/diethyl ether. There was obtained 355 mg (60%) of the title compound as a white powder. Melting point: 220.7° C. [α]²⁰_{D}=−199.6 (c, 0.010 g/mL, MeOH). ¹H NMR (800 MHz, methanol-d₄): δ 1.07 (t, 3H), 1.40 (m, 1H), 1.77 (m, 1H), 1.86 (m, 3H), 1.9-2.1 (m, 4H), 2.4-2.5 (m, 4H), 2.56 (m, 1H), 2.68 (m, 1H), 2.92 (m, 1H), 3.10 (m, 2H), 3.23 (m, 1H), 3.32 (m, 1H), 3.62 (m, 1H).

### Example 4B

Epimer 1 of (4aR,10aR)-1-propyl-1,2,3,4,4a,5,6,7,8,9,10,10a-dodecahydrobenzo[g]quinolin-6-ol (4aR,10aR)-1-propyl-1,2,3,4,4a,5,8,9,10,10a-decahydrobenzo[g]quinolin-6(7H)-one in its non-salt form from Preparation 4A (0.33 g, 1.33 mmol) was dissolved in MeOH (8 mL) and cerium (Ill) chloride heptahydrate (596 mg, 1.6 mmol) was added at 0° C. The mixture was stirred for 15 min with cooling and then NaBH₄ was added in three portions during 15 min. The reaction mixture was stirred for an additional hour with cooling and then water (10 mL) was added. After stirring for 20 min, the mixture was extracted with EtOAc (4×50 mL). The combined organic solutions were evaporated and the two epimers were separated by repeated silica-gel chromatography using EtOAc as eluent. There was obtained 144 mg (43%) of epimer 1 of (4aR,10aR)-1-propyl-1,2,3,4,4a,5,6,7,8,9,10,10a-dodecahydrobenzo[g]quinolin-6-ol as an off-white powder. ¹H NMR (800 MHz, CDCl₃): δ 0.86 (t, 3H), 1.02 (qd, 1H), 1.3-1.7 (m, 7H), 1.76 (m, 2H), 1.91 (m, 5H), 2.02 (m, 2H), 2.21 (m, 2H), 2.35 (td, 1H), 2.65 (td, 1H), 2.95 (d, 1H), 4.01 (d, 1H). ¹³C NMR (201 MHz, CDCl₃): δ 12.1 (s), 17.5 (s), 19.3 (s), 25.6 (s), 30.5 (s), 32.0 (s), 32.7 (s), 35.1 (s), 36.5 (s), 37.8 (s), 52.9 (s), 55.5 (s), 61.2 (s), 70.0 (s), 129.3 (s), 130.9 (s).

### Example 5

### Synthesis of oxalic acid salt of (4aR,10aR)-1-propyl-1,2,3,4,4a,5,6,7,8,9,10,10a-dodecahydrobenzo[g]quinolin-6-yl acetate (Mixture of 6R and 6S epimers)

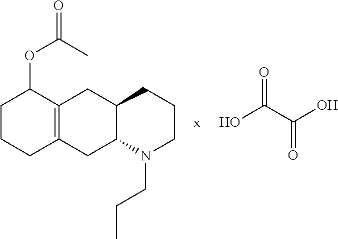 [SMILES: CCCN1CCC[C@@H]2CC3=C(CCCC3OC(C)=O)C[C@H]21 ; O=C(O)C(=O)O]

An isomeric mixture of the 6R and 6S epimers of (4aR,10aR)-1-propyl-1,2,3,4,4a,5,6,7,8,9,10,10a-dodecahydrobenzo[g]quinolin-6-ol obtained in a similar fashion as in Example 2 (82 mg, 0.33 mmol) was dissolved in a mixture of pyridine (1 mL) and acetic anhydride (1 mL). The reaction mixture was stirred at 30-35° C. for 4 h and then quenched by the addition of ethanol at room temperature. The volatiles were removed by evaporation and co-evaporation several times with ethanol. The residue was dissolved in ethanol (3 mL) together with oxalic acid dihydrate (45 mg, 0.35 mmol). After efforts were made to obtain the product as a crystalline salt by the addition of ether, the solvents were removed by evaporation. There was obtained 128 mg of an isomeric mixture of the desired compound as an oil. MS (ESI+) m/z 292 [M+H]⁺. ¹H NMR (700 MHz, DMSO-d₆) δ 5.0-5.2 (m, 1H), 3.11 (m, 2H), 2.94 (m, 2H), 2.4-2.5 (m, 2H), 2.23 (m, 1H), 2.00 and 2.02 (two singlets, 1H), 1.5-2.0 (m, 14H), 1.22 (m, 1H), 0.92 (t, 3H).

### Example 6

### Synthesis of D-tartaric acid salt of (4aR,6R,10aR)-1-propyl-1,2,3,4,4a,5,6,7,8,9,10,10a-dodecahydrobenzo[g]quinolin-6-yl acetate

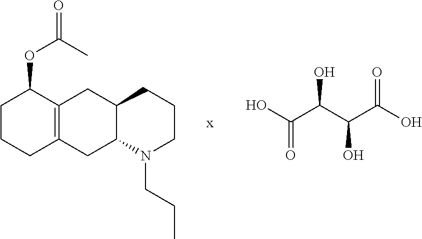 [SMILES: CCCN1CCC[C@@H]2CC3=C(CCC[C@H]3OC(C)=O)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (500 mg, 2.0 mmol), was dissolved in a mixture of pyridine (4 mL) and acetic anhydride (4 mL). The reaction mixture was placed in a fume hood overnight and then quenched by the addition of ethanol at room temperature. After 30 minutes, the volatiles were removed by evaporation and co-evaporation several times using ethanol. The residue was purified by silica-gel chromatography using a gradient of EtOAc and MeOH (0-70% MeOH) as eluent. The fractions were analyzed by TLC (silica-gel) where the spots were made visible with iodine. The Rf-value of the ester was 0.35 (MeOH/EtOAc, 1:1) and the Rf-value of the starting material was 0.23. The desired fractions were evaporated and the residue (330 mg) was dissolved in EtOAc (9 mL) in a round bottom flask. To the formed solution was added D-tartaric acid (170 mg, 1.1 mmol) and after stirring at room temperature for 24 h, the mixture became two immiscible liquids. A part of the sticky oil at the bottom of the flask was collected by a spatula and transferred to a test tube together with fresh EtOAc. After persistently scratching the oil with a glass stick at the inner surface of the tube, crystals eventually were formed and these were then transferred to the round bottom flask containing the main mixture. The flask was provided with a stopper and was placed in a fume hood for further 24 h and the formed precipitate was isolated by filtration. The filter cake was washed with EtOAc and then dried under vacuum. There was obtained 270 mg (30%) of the desired D-tartrate salt as a white powder. Melting point: 83.6° C. [α]_{D}=−46.5° (c 10 mg/mL, MeOH). MS (ESI+) m/z 292 [M+H]⁺. ¹H NMR (800 MHz, DMSO-d₆) δ 5.13 (d, 1H), 4.04 (s, 2H), 3.29 (d, 1H), 2.98 (m, 1H), 2.7-2.8 (m, 3H), 2.40 (m, 1H), 2.16 (m, 1H), 2.00 (s, 3H), 1.8-2.0 (m, 5H), 1.73 (m, 4H), 1.65 (m, 2H), 1.55 (m, 3H), 1.15 (m, 1H), 0.89 (t, 3H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl acetate is provided as a combination of (4aR,10aR)-1-propyl-1,2,3,4,4a,5,6,7,8,9,10,10a-dodecahydrobenzo[g]quinolin-6-yl acetate and D-tartaric acid taken in a ratio of 1:1.

### Example 7

### Synthesis of D-tartaric acid salt of (4aR,6R,10aR)-1-propyl-1,2,3,4,4a,5,6,7,8,9,10,10a-dodecahydrobenzo[g]quinolin-6-yl isobutyrate

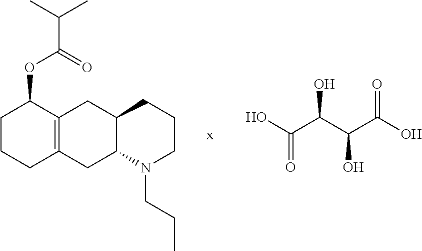 [SMILES: CCCN1CCC[C@@H]2CC3=C(CCC[C@H]3OC(=O)C(C)C)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (113 mg, 0.45 mmol), was dissolved in DCM (7 mL) and to the solution were added triethylamine (0.20 mL, 1.4 mmol) and isobutyryl chloride (0.10 mL, 0.95 mmol) in the given order. The reaction mixture was stirred overnight at room temperature and then diluted with DCM. After washing the solution with aqueous Na₂CO₃ (5%), the organic phase was filtered through a phase-separator and the volatiles were removed by evaporation. The residue was purified by silica-gel chromatography using a gradient of EtOAc and MeOH (0-70% MeOH) as eluent. The fractions were analyzed by TLC (silica-gel) where the spots were made visible with iodine. The Rf-value of the ester was 0.38 (MeOH/EtOAc, 1:1) and the Rf-value of the starting material was 0.23. The desired fractions were evaporated and the residue (125 mg) was dissolved in EtOAc (10 mL) in a screw top bottle (25 mL) whereupon the free amine started to crystallize. To the slurry was added D-tartaric acid (59 mg, 0.39 mmol) and the mixture was heated on a water bath in an attempt to have most of the material in solution. The bottle without a screw cap was placed in a fume hood at room temperature for three days whereupon the salt started to precipitate. After a further four days at room temperature with the screw cap closed, the salt was isolated by filtration. The filter cake was washed with EtOAc and then dried under vacuum. There was obtained 106 mg (49%) of the desired D-tartrate salt as a white powder. Melting point: 79.1° C. [α]_{D}=−72.0° (c 1.0 mg/mL, MeOH). MS (ESI+) m/z 320 [M+H]⁺. ¹H NMR (700 MHz, DMSO-d₆) δ 5.13 (d, 1H), 4.02 (s, 2H), 3.25 (d, 1H), 2.95 (m, 1H), 2.72 (m, 3H), 2.49 (m, 1H), 2.40 (m, 1H), 2.12 (m, 1H), 1.5-2.0 (m, 14H), 1.13 (m, 1H), 1.08 (m, 6H), 0.89 (t, 3H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl 2-methylpropionate is provided as a combination of (4aR,10aR)-1-propyl-1,2,3,4,4a,5,6,7,8,9,10,10a-dodecahydrobenzo[g]quinolin-6-yl 2-methylpropionate and D-tartaric acid taken in a ratio of 1:1.

### Example 8

### Synthesis of D-tartaric acid salt of (4aR,6R,10aR)-1-propyl-1,2,3,4,4a,5,6,7,8,9,10,10a-dodecahydrobenzo[g]quinolin-6-yl cyclopropanecarboxylate

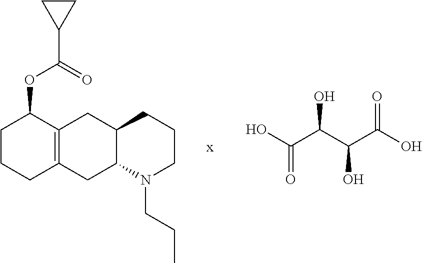 [SMILES: CCCN1CCC[C@@H]2CC3=C(CCC[C@H]3OC(=O)C3CC3)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (113 mg, 0.45 mmol), was dissolved in DCM (7 mL) and to the formed solution were added triethylamine (0.30 mL, 2.2 mmol) and cyclopropanecarbonyl chloride (0.20 mL, 2.2 mmol) in the given order. The reaction mixture was stirred overnight at room temperature and then diluted with DCM. After washing the solution with aqueous Na₂CO₃ (5%), the organic phase was filtered through a phase-separator and the volatiles were removed by evaporation. The residue was purified by silica-gel chromatography using a gradient of EtOAc and MeOH (0-70% MeOH) as eluent and the fractions were analyzed by TLC (silica-gel) where the spots were made visible with iodine. The Rf-value of the ester was 0.38 (MeOH/EtOAc, 1:1) and the Rf-value of the starting material was 0.23. The desired fractions were evaporated and the residue (110 mg) was dissolved in EtOAc (15 mL) in a screw top bottle (25 mL). To the solution was added D-tartaric acid (55 mg, 0.37 mmol) and the mixture was heated on a water bath in an attempt to have most of the material in solution. The bottle without screw cap was placed in a fume hood at room temperature for one day and the formed salt was isolated by filtration. The solid material was recrystallized from hot EtOAc and the salt was dried under vacuum. There was obtained 65 mg (30%) of the desired D-tartrate salt as a white powder. Melting point: 73.3° C. [α]_{D}=−42.0° (c 1.0 mg/mL, MeOH). MS (ESI+) m/z 318 [M+H]⁺. ¹H NMR (700 MHz, DMSO-d₆) δ 5.15 (d, 1H), 4.02 (s, 2H), 3.25 (d, 1H), 2.95 (m, 1H), 2.6-2.7 (m, 3H), 2.42 (m, 1H), 2.11 (m, 1H), 1.5-2.0 (m, 15H), 1.15 (m, 1H), 0.8-0.9 (m, 7H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl cyclopropanecarboxylate is provided as a combination of (4aR,10aR)-1-propyl-1,2,3,4,4a,5,6,7,8,9,10,10a-dodecahydrobenzo[g]quinolin-6-yl cyclopropanecarboxylate and D-tartaric acid taken in a ratio of 1:1.

### Example 9

### Synthesis of D-tartaric acid salt of (4aR,6R,10aR)-1-propyl-1,2,3,4,4a,5,6,7,8,9,10,10a-dodecahydrobenzo[g]quinolin-6-yl 2,2-dimethylpropionate

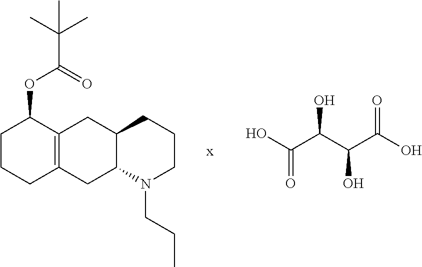 [SMILES: CCCN1CCC[C@@H]2CC3=C(CCC[C@H]3OC(=O)C(C)(C)C)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (170 mg, 0.68 mmol), was dissolved in DCM (10 mL) and to the solution were added triethylamine (0.30 mL, 2.1 mmol) and pivaloyl chloride (0.20 mL, 1.63 mmol) in the given order. The reaction mixture was stirred at room temperature for three days and then diluted with DCM. After washing the solution with aq. Na₂CO₃ (5%), the organic phase was filtered through a phase-separator and the volatiles were removed by evaporation. The residue was purified by silica-gel (25 g) flash chromatography using a gradient of EtOAc and MeOH (0-65% MeOH) as eluent. The desired fractions were evaporated, and the residue (168 mg) was dissolved in EtOAc (10 mL) together with D-tartaric acid (80 mg, 0.53 mmol). The mixture was stirred at room temperature for three days without any solid being precipitated. After further five weeks at room temperature without stirring with the flask closed with a stopper, a salt precipitated, and the solid was isolated by filtration. The filter cake was washed with EtOAc and then dried under vacuum. There was obtained 168 mg (40%) of the desired D-tartrate salt as a white powder. Melting point: 82.0° C. [α]_{D}=−70.0° (c 1.0 mg/mL, MeOH). MS (ESI+) m/z 334 [M+H]⁺. ¹H NMR (700 MHz, DMSO-d6) δ 5.11 (s, 1H), 4.03 (s, 2H), 3.26 (d, 1H), 2.96 (m, 1H), 2.70 (m, 3H), 2.41 (m, 1H), 2.40 (m, 1H), 2.13 (m, 1H), 1.5-2.0 (m, 13H), 1.13 (s, 9H), 1.12 (m, 1H), 0.89 (t, 3H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl 2,2-dimethylpropionate is provided as a combination of (4aR,10aR)-1-propyl-1,2,3,4,4a,5,6,7,8,9,10,10a-dodecahydrobenzo[g]quinolin-6-yl 2,2-dimethylpropionate and D-tartaric acid taken in a ratio of 1:1.

### Example 10

### Synthesis of D-tartaric acid salt of (4aR,6R,10aR)-1-propyl-1,2,3,4,4a,5,6,7,8,9,10,10a-dodecahydrobenzo[g]quinolin-6-yl cyclobutanecarboxylate

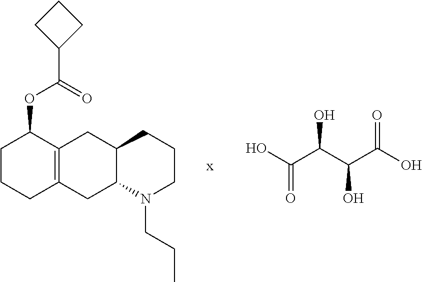 [SMILES: CCCN1CCC[C@@H]2CC3=C(CCC[C@H]3OC(=O)C3CCC3)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (156 mg, 0.63 mmol), was dissolved in dry DCM (10 mL) and to the solution was added triethylamine (0.30 mL, 2.2 mmol) followed by dropwise addition of cyclobutanecarbonyl chloride (0.20 mL, 1.7 mmol). The reaction mixture was stirred at room temperature for four days and then diluted with DCM. After washing with aq. Na₂CO₃ (5%), the aq. phase was extracted with DCM and the combined organic phases were washed with aq. Na₂CO₃ (5%) and then passed through a phase-separator. The volatiles were removed by evaporation and the residue (231 mg) was purified by silica-gel (25 g) flash chromatography using a gradient of EtOAc and MeOH (0-67% MeOH) as eluent. The fractions were analyzed by TLC (silica-gel) where the spots were made visible with iodine. The Rf-value of the ester was 0.34 (MeOH/EtOAc, 1:1). After evaporation of the desired fractions, there was obtained 141 mg of a pinkish oil that was dissolved in EtOAc (15 mL). The solution was filtered through a glass filter funnel. To the filtrate was added D-tartaric acid (66 mg, 0.44 mmol) while stirring. The mixture was stirred at room temperature for 20 h. The stirring was stopped and the round bottom flask, which was closed with a stopper, was left in the hood for six days. A small amount of an oily precipitate at the bottom of the flask was triturated with a glass rod until a crystallization was initiated. The formed solid was collected by filtration and then washed with a small amount of EtOAc. After drying in vacuum at 40° C., there was obtained 142 mg (47%) of the desired D-tartrate salt as a pale pink powder. Melting point: 60.2° C. [α]_{D}=−49.7° (c 1.0 mg/mL, MeOH). HRMS: m/z (M+H)⁺ calculated for C₂₁H₃₄NO₂: 332.2589, found 332.2589.

¹H NMR (800 MHz, DMSO-d6) δ 5.15 (s, 1H), 4.02 (s, 2H), 3.25 (d, 1H), 3.13 (m, 1H), 2.95 (m, 1H), 2.70 (m, 3H), 2.39 (m, 1H), 2.14 (m, 5H), 1.6-2.0 (m, 13H), 1.54 (m, 3H), 1.11 (m, 1H), 0.89 (t, 3H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl cyclobutanecarboxylate is provided as a combination of (4aR,10aR)-1-propyl-1,2,3,4,4a,5,6,7,8,9,10,10a-dodecahydrobenzo[g]quinolin-6-yl cyclobutanecarboxylate and D-tartaric acid taken in a ratio of 1:1.

### Example 11

### Synthesis of D-tartaric acid salt of (4aR,6R,10aR)-1-propyl-1,2,3,4,4a,5,6,7,8,9,10,10a-dodecahydrobenzo[g]quinolin-6-yl 1-methylcyclopropanecarboxylate

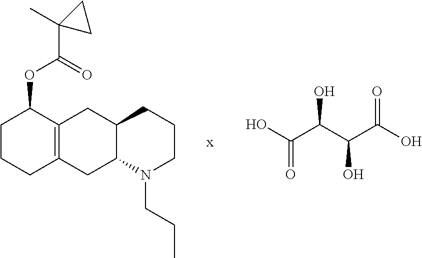 [SMILES: CCCN1CCC[C@@H]2CC3=C(CCC[C@H]3OC(=O)C3(C)CC3)C[C@H]21 ; O=C(O)[C@@H](O)[C@H](O)C(=O)O]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (150 mg, 0.60 mmol), was dissolved in DCM (10 mL). Triethylamine (0.25 mL, 1.8 mmol) and 1-methylcyclopropanecarbonyl chloride (0.18 mL, 1.5 mmol) were added in the given order. The reaction mixture was stirred under nitrogen at room temperature for three days and then another portion of triethylamine (0.25 mL, 1.8 mmol) as well as another portion of 1-methylcyclopropanecarbonyl chloride (0.18 mL, 1.5 mmol) was added. After stirring four additional four days, further portions of triethylamine (0.25 mL, 1.8 mmol) and 1-methylcyclopropanecarbonyl chloride (0.18 mL, 1.5 mmol) were added. The mixture was stirred for additional two days and then diluted with DCM. After washing with aq. Na₂CO₃ (5%), the aq. phase was extracted twice with DCM and the combined organic solutions were washed with brine and dried over Na₂SO₄. The volatiles were removed by evaporation. The residue was purified by silica-gel (25 g) flash chromatography using a gradient of EtOAc and MeOH (0-80% MeOH). The desired fractions were evaporated and a light red/brown solid (75 mg) was obtained. The solid was almost completely dissolved in hot EtOAc (15 mL). D-tartaric acid (36 mg, 0.24 mmol) was added and the mixture was heated on a water bath until all the material was dissolved. The round bottom flask was equipped with a vacuum adapter in order to slowly evaporate the solvent on allowing the flask to stand in the hood. After two weeks no solid had precipitated and the residue was dissolved in water. The water was removed by freeze-drying and there was obtained 60 mg (55%) of the desired D-tartrate salt as a light brown solid. MS (ESI+) m/z 332 [M+H]⁺. ¹H NMR (700 MHz, DMSO-d₆) δ 5.11 (d, 1H), 4.14 (s, 2H), 3.36 (d, 1H), 3.05 (m, 1H), 2.98 (m, 1H), 2.82 (m, 2H), 2.45 (m, 1H), 2.24 (m, 1H), 1.4-2.0 (m, 13H), 1.21 (s, 3H), 1.20 (m, 1H), 1.06 (m, 2H), 0.91 (t, 3H), 0.74 (m, 2H). The NMR spectrum showed a ratio of 1:1 between the above-identified ester and D-tartaric acid. Thus, in this example the D-tartaric acid salt of (4aR,10aR)-1-propyl-1,2,3,4,4a,5,6,7,8,9,10,10a-dodecahydrobenzo[g]quinolin-6-yl 1-methylcyclopropanecarboxylate is provided as a combination of (4aR,10aR)-1-propyl-1,2,3,4,4a,5,6,7,8,9,10,10a-dodecahydrobenzo[g]quinolin-6-yl 1-methylcyclopropanecarboxylate and D-tartaric acid taken in a ratio of 1:1.

### Example 12

### Preparation 1

Epimer 1 of (4aR,10aR)-1-ethyl-1,2,3,4,4a,5,6,7,8,9,10,10a-dodecahydrobenzo[g]quinolin-6-ol

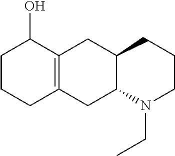 [SMILES: CCN1CCC[C@@H]2CC3=C(CCCC3O)C[C@H]21]

### A) 3-(4-Methoxyphenyl)propanoyl chloride

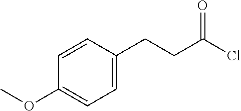

3-(4-Methoxyphenyl)propionic acid (50.0 g, 272 mmol) was dissolved in DCM (500 mL) and to the formed solution was added thionylchloride (49.3 mL, 680 mmol). The mixture was heated to reflux for 6 h, allowed to cool to room temperature and then evaporated to dryness using a rotary evaporator. The crude acid chloride (52.8 g, 98%) was used in the next step without further purification.

### B) N-Ethyl-3-(4-methoxyphenyl)propanamide

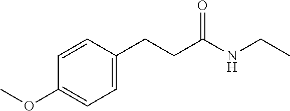

3-(4-Methoxyphenyl)propanoyl chloride (23.9 g, 120 mmol) was dissolved in dry THF (30 mL) and the formed solution was slowly added to an ice-cold mixture of ethylamine (2 M in THF, 150 mL, 300 mmol) and triethylamine (20 mL, 144 mmol) in THF (80 mL). The reaction mixture was stirred at room temperature for one hour and then diluted with aq. sodium carbonate (10%). The mixture was extracted several times with EtOAc, and the combined organic solutions were washed with brine and dried over sodium sulphate. The solvent was removed by evaporation and there was obtained 19.0 g (76%) of the desired amide which was used in the next step without further purification. GC MS m/z (relative intensity, 70 eV) 208 (10), 207 (71), 135 (23), 134 (79), 122 (9), 121 (bp), 119 (9), 108 (9), 91 (19), 78 (11), 77 (14).

### C) N-Ethyl-3-(4-methoxyphenyl)propan-1-amine

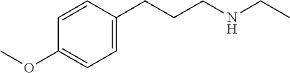

N-ethyl-3-(4-methoxyphenyl)propenamide (19.0 g, 91.7 mmol) was dissolved in dry THF (150 mL) and the formed solution was added dropwise to a mixture of LiAlH₄ (6.96 g, 183 mmol) in THF (120 mL). The reaction mixture was heated to reflux for 3.5 h, cooled with an ice-bath and then diluted with THF (150 mL). After successively quenching with water (7 mL), aq. NaOH (15%, 7 mL) and finally with water (21 mL), the mixture was stirred for 15 min and then the solids were removed by filtration. The filter cake was washed with EtOH (3×20 mL) and the filtrate was evaporated to dryness using a rotary evaporator. Water (50 mL) was added to the residue and the mixture was extracted several times with EtOAc. The combined organic solutions were washed with brine and then dried over sodium sulphate. The solvent was removed by evaporation and there was obtained 16.8 g (95%) of the desired amine that was used in the next step without further purification. GC MS m/z (relative intensity, 70 eV) 193 (38), 148 (51), 147 (20), 134 (7), 121 (30), 117 (7), 91 (15), 78 (11), 77 (13), 70 (7), 58 (bp).

### D) (4aR,8aR)-1-ethyloctahydroquinolin-7(1H)-one

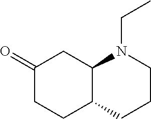

N-Ethyl-3-(4-methoxyphenyl)propylan-1-amine (16.8 g, 87.0 mmol) was dissolved in dry THF (180 mL) in a three necked round bottom flask and the solution was flushed with nitrogen for several minutes before t-butanol (16 mL, 192 mmol) was added. The solution was cooled to −60° C. and then anhydrous ammonia was added via the gas inlet with continues cooling until the volume of the reaction mixture had increased by 180 mL. Metallic lithium (2.35 g, 295 mmol) was slowly added in small portions and the mixture was stirred at −60° C. for 4 h. A mixture of MeOH and saturated aq. ammonium chloride (1:1, 62 mL) was added to the mixture which then was allowed to warm to room temperature. After carefully heating with a water bath until most of the ammonia had been evaporated from the flask, the pH was adjusted to about 1 by the addition of concentrated hydrochloric acid. The mixture was stirred at room temperature for 18 h and then the pH was adjusted to above 9 at a temperature below 15° C. by the addition of aq. 4 M NaOH. After extracting the basic mixture several times with DCM, the combined organic solutions were washed with brine and then dried over sodium sulphate. After the solvent was removed by evaporation, the product was purified (and the two stereoisomers were separated) by silica-gel chromatography using EtOAc/MeOH (gradient, 0-10% MeOH) as eluent. There was obtained 4.66 g of the first eluting cis isomer and then 0.51 g of the trans isomer, respectively. The cis isomer was then converted into the desired trans isomer by dissolving 4.66 g in ethanolic KOH (1%, 470 mL), stirring the formed solution at room temperature for four days under a nitrogen atmosphere and the flask covered with aluminium foil. After performing a similar work-up and separation procedures as described above, there was obtained 3.25 g (20%) in total of the racemic trans isomer. GC MS m/z (relative intensity, 70 eV) 181 (14), 166 (7), 125 (10), 124 (bp), 111 (6), 110 (6), 96 (13), 56 (4), 55 (4). ¹H NMR (800 MHz, CDCl₃) δ 2.94 (dq, 1H), 2.81 (m, 1H), 2.78 (m, 1H), 2.53 (dq, 1H), 2.37 (m, 2H), 2.26 (t, 1H), 2.20 (td, 1H), 2.09 (ddd, 1H), 1.90 (ddt, 1H), 1.81 (dt, 1H), 1.71 (m, 2H), 1.62 (ddd, 1H), 1.36 (tdd, 1H), 1.05 (m, 1H), 0.99 (t, 3H).

### E) (E)-ethyl 4-((4aS,8aR)-1-ethyloctahydroquinolin-7(1H)-ylidene)butanoate

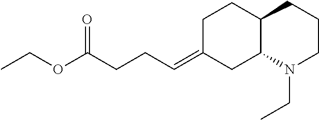 [SMILES: CCOC(=O)CC/C=C1\CC[C@H]2CCCN(CC)[C@@H]2C1]

[3-(Ethoxycarbonyl)propyl]triphenylphosphonium bromide (16.7 g, 35.9 mmol) was dissolved in dry DMF (55 mL) and the formed solution was added dropwise to a cooled (0° C.) solution of potassium tert-butoxide (4.1 g, 35.9 mmol) in DMF (6 mL) under a nitrogen atmosphere. The mixture was stirred with cooling for 30 min. and then a solution of (4aR,8aR)-1-ethyloctahydroquinolin-7(1H)-one (3.25 g, 17.9 mmol) in DMF (7 mL) was added dropwise at 0° C. The reaction mixture was stirred with cooling for 4 h and then for 18 h at room temperature. After cooling with an ice-bath, water (120 mL) was added, and the product was extracted several times with diethyl ether. The organic solutions were successively washed with aq. LiCl (5%, 75 mL) and brine, dried over sodium sulphate and concentrated to dryness on a rotary evaporator. The residue, which consisted of a mixture of the desired ethyl ester and the corresponding tert-butyl ester as a by-product, was dissolved in ethanol (50 mL) together with concentrated sulfuric acid (1 mL). The mixture was heated to reflux for 18 h and then allowed to cool to room temperature. The solvent was removed by evaporation and the residue diluted with water. The pH was adjusted to over 10 by the addition of saturated aq. Na₂CO₃ and then the mixture was extracted several times with EtOAc. The combined organic solutions were washed with brine, dried over sodium sulphate, and then evaporated. The product was purified by silica-gel chromatography using isooctane/EtOAc/MeOH (gradient, 0-100% EtOAc and then 0-100% MeOH) as eluent. There was obtained 4.68 g (93%) of the desired ethyl ester as an oil. GC MS m/z (relative intensity, 70 eV) 279 (4), 234 (5), 125 (10), 124 (bp), 111 (2), 110 (2), 96 (5), 91 (2), 79 (2).

### F) (4aR,10aR)-1-ethyl-1,2,3,4,4a,5,8,9,10,10a-decahydrobenzo[g]quinolin-6(7H)-one

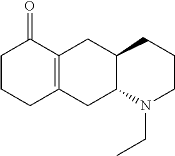 [SMILES: CCN1CCC[C@@H]2CC3=C(CCCC3=O)C[C@H]21]

(E)-ethyl 4-((4aS,8aR)-1-ethyloctahydroquinolin-7(1H)-ylidene)butanoate (4.67 g, 16.7 mmol) was mixed with Eaton's reagent (18 mL, Phosphorus pentoxide in methanesulfonic acid, 7.7%). The reaction mixture was stirred overnight at 70° C., allowed to cool to room temperature, and then carefully poured into aq. Na₂CO₃ (10%, 300 mL). After extracting the basic mixture several times with DCM, the combined organic solutions were washed with brine, dried over sodium sulphate, and then concentrated to dryness using a rotary evaporator. The residue was purified by silica-gel chromatography using EtOAc/MeOH (gradient, 0-30% MeOH) as eluent and there was obtained 1.86 g (48%) of the racemic product as an oil. Separating the two enantiomers by repetitive chiral chromatography (Chiralpak® IG, 250×20 mm) using heptane, IPA and DEA (60:40:0.1) as eluent afforded the (+)-enantiomer as the first eluting isomer and the (−)-enantiomer as the last eluting isomer, respectively (approximately 45 mg of the racemic mixture was injected on the column each time). In total, there was obtained 808 mg of the desired (−)-isomer of (4aR,10aR)-1-ethyl-1,2,3,4,4a,5,8,9,10,10a-decahydrobenzo[g]quinolin-6(7H)-one in its non-salt form as an oil consisting of 99.5% of the (−)-isomer and 0.5% of the (+)-isomer as determined by analytical chiral chromatography. Also, there was obtained 795 mg of the (+)-isomer of said compound in its non-salt form as an oil.

Data for the (−)-isomer of (4aR,10aR)-1-ethyl-1,2,3,4,4a,5,8,9,10,10a-decahydrobenzo[g]quinolin-6(7H)-one in its non-salt form: MS (ESI+) m/z 234 [M+H]⁺. [α]_{D}²⁵=−199° (c=0.05, methanol). ¹H NMR (800 MHz, CDCl₃) δ 2.95 (m, 1H), 2.87 (m, 1H), 2.53 (m, 3H), 2.42 (m, 1H), 2.36 (m, 1H), 2.26 (m, 2H), 2.18 (m, 2H), 2.05 (td, 1H), 1.97 (m, 2H), 1.85 (m, 1H), 1.68 (m, 3H), 1.41 (m, 1H), 1.05 (m, 4H). The absolute configuration of the two enantiomers was not determined by x-ray crystallography but by comparing the sign of optical rotation as well as elution order on the chiral column with that of the propyl analogue according to Example 4a. Thus, it was concluded that the (−)-isomer has the (4aR,10aR)-configuration and the (+)-isomer has the (4aS,10aS)-configuration.

### G) Epimer 1 of (4aR,10aR)-1-ethyl-1,2,3,4,4a,5,6,7,8,9,10,10a-dodecahydrobenzo[g]quinolin-6-ol

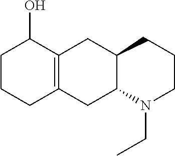 [SMILES: CCN1CCC[C@@H]2CC3=C(CCCC3O)C[C@H]21]

(4aR,10aR)-1-ethyl-1,2,3,4,4a,5,8,9,10,10a-decahydrobenzo[g]quinolin-6(7H)-one (0.68 g, 2.9 mmol) was dissolved in MeOH (20 mL) and to the formed solution was added cerium (III) chloride heptahydrate (1.3 g, 3.5 mmol) at 0° C. The mixture was stirred with cooling for 15 min. Sodium borohydride (0.33 g, 8.8 mmol) was added in three portions over a period of 15 min and the reaction mixture was stirred for 1 h. Water (30 mL) was added and then stirring was continued for 20 min. After extracting the mixture seven times with EtOAc, the combined organic solutions were washed with brine, dried over sodium sulphate, and then concentrated to dryness using a rotary evaporator. The residue was purified by silica-gel chromatography using EtOAc/MeOH (gradient, 0-50% MeOH) as eluent and there was obtained 0.14 g (16%) of the first eluting isomer as an oil. Also, there was obtained 43 mg of epimer 2 as an oil. Data for epimer 1 of (4aR,10aR)-1-ethyl-1,2,3,4,4a,5,6,7,8,9,10,10a-dodecahydrobenzo[g]quinolin-6-ol: MS (ESI+) m/z 236 [M+H]⁺. ¹H NMR (800 MHz, CDC₃) δ 4.02 (q, 1H), 2.92 (m, 1H), 2.84 (m, 1H), 2.55 (m, 1H), 2.22 (m, 2H), 1.5-2.1 (m, 14H), 1.04 (m, 1H), 1.00 (m, 3H).

### Preparation 2

### Epimer 1 of (4aR,10aR)-1-methyl-1,2,3,4,4a,5,6,7,8,9,10,10a-dodecahydrobenzo[g]quinolin-6-ol

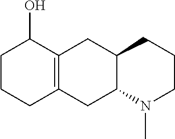 [SMILES: CN1CCC[C@@H]2CC3=C(CCCC3O)C[C@H]21]

### A) (4aR,8aR)-1-methyloctahydroquinolin-6(2H)-one

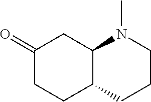

(4aR,8aR)-1-methyloctahydroquinolin-6(2H)-one was synthesized in a similar fashion as in Preparation 1a-1d but using methylamine rather than ethylamine as starting material. Starting with 22 g (111 mmol) of 3-(4-methoxyphenyl)propanoyl chloride and 150 mL of methylamine in THF (2M, 300 mmol) afforded 1 g (6%) of (4aR,8aR)-1-methyloctahydroquinolin-6(2H)-one as an oil. GC MS m/z (relative intensity, 70 eV) 168 (2), 167 (14), 166 (5), 124 (3), 111 (10), 110 (bp), 108 (2), 97 (10), 96 (11), 95 (2), 94 (2), 82 (5), 81 (2), 70 (4), 68 (3), 67 (3), 55 (4), 54 (3), 53 (2). ¹H NMR (800 MHz, CDCl₃) δ 2.90 (m, 1H), 2.81 (m, 1H), 2.2-2.4 (m, 4H), 2.06 (m, 1H), 1.90 (m, 1H), 1.7-1.8 (m, 4H), 1.60 (m, 1H), 1.35 (tdd, 1H), 1.06 (m, 1H).

### B) (E)-ethyl 4-((4aS,8aR)-1-methyloctahydroquinolin-7(1H)-ylidene)butanoate

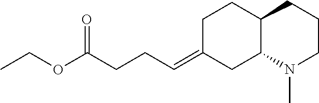 [SMILES: CCOC(=O)CC/C=C1\CC[C@H]2CCCN(C)[C@@H]2C1]

(E)-ethyl 4-((4aS,8aR)-1-methyloctahydroquinolin-7(1H)-ylidene)butanoate was synthesized in a similar fashion as in 5E but (4aR,8aR)-1-methyloctahydroquinolin-6(2H)-one rather than (4aR,8aR)-1-ethyloctahydroquinolin-6(2H)-one as starting material. Starting with 2.3 g (14 mmol) of (E)-ethyl 4-((4aS,8aR)-1-methyloctahydroquinolin-7(1H)-ylidene)butanoate afforded 3.6 g (100%) of (E)-ethyl 4-((4aS,8aR)-1-methyloctahydroquinolin-7(1H)-ylidene)butanoate as an oil.

### C) (4aR,10aR)-1-methyl-1,2,3,4,4a,5,8,9,10,10a-decahydrobenzo[g]quinolin-6(7H)-one

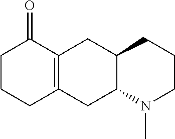 [SMILES: CN1CCC[C@@H]2CC3=C(CCCC3=O)C[C@H]21]

The (−)-isomer of (4aR,10aR)-1-methyl-1,2,3,4,4a,5,8,9,10,10a-decahydrobenzo[g]quinolin-6(7H)-one was synthesized in a similar fashion as in 5F but using (E)-ethyl 4-((4aS,8aR)-1-methyloctahydroquinolin-7(1H)-ylidene)butanoate rather than (E)-ethyl 4-((4aS,8aR)-1-ethyloctahydroquinolin-7(1H)-ylidene)butanoate. Starting with 3.2 g (12 mmol) of (E)-ethyl 4-((4aS,8aR)-1-methyloctahydroquinolin-7(1H)-ylidene)butanoate afforded 0.53 g (20%) of the (−)-isomer of (4aR,10aR)-1-methyl-1,2,3,4,4a,5,8,9,10,10a-decahydrobenzo[g]quinolin-6(7H)-one as an oil.

MS (ESI+) m/z 220 [M+H]⁺. [α]_{D}²⁵=−244° (c=0.05, methanol). ¹H NMR (800 MHz, CDCl₃) δ 2.88 (m, 1H), 2.53 (m, 3H), 2.42 (m, 1H), 2.36 (m, 1H), 2.27 (m, 2H), 2.14 (m, 2H), 1.97 (m, 2H), 1.85 (m, 1H), 1.74 (m, 1H), 1.6-1.7 (m, 3H), 1.40 (m, 1H), 1.06 (m, 1H). The absolute configuration of the two enantiomers was not determined by x-ray crystallography but by comparing the sign of optical rotation as well as elution order on the chiral column with that of the propyl analogue according to Example 4a. Thus, it was concluded that the (−)-isomer has the (4aR,10aR)-configuration and the (+)-isomer has the (4aS,10aS)-configuration.

### D) Epimer 1 of (4aR,10aR)-1-methyl-1,2,3,4,4a,5,6,7,8,9,10,10a-dodecahydrobenzo[g]quinolin-6-ol

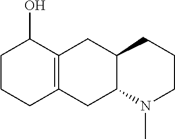 [SMILES: CN1CCC[C@@H]2CC3=C(CCCC3O)C[C@H]21]

Epimer 1 of (4aR,10aR)-1-methyl-1,2,3,4,4a,5,6,7,8,9,10,10a-dodecahydrobenzo[g]quinolin-6-ol was synthesized in a similar fashion as in 5G but using (4aR,10aR)-1-methyl-1,2,3,4,4a,5,8,9,10,10a-decahydrobenzo[g]quinolin-6(7H)-one rather than (4aR,10aR)-1-ethyl-1,2,3,4,4a,5,8,9,10,10a-decahydrobenzo[g]quinolin-6(7H)-one. Starting with 460 mg (2 mmol) of (4aR,10aR)-1-methyl-1,2,3,4,4a,5,8,9,10,10a-decahydrobenzo[g]quinolin-6(7H)-one afforded 0.136 mg (29%) of the epimer 1 of (4aR,10aR)-1-methyl-1,2,3,4,4a,5,6,7,8,9,10,10a-dodecahydrobenzo[g]quinolin-6-ol as an oil. MS (ESI+) m/z 222 [M+H]⁺. ¹H NMR (800 MHz, CDCl₃) δ 4.01 (t, 1H), 2.25 (s, 3H), 2.23 (m, 1H), 2.09 (td, 1H), 2.00 (m, 1H), 1.95 (m, 1H), 1.90 (m, 4H), 1.4-1.8 (m, 10H), 1.04 (m, 1H).

### Example 13

### Synthesis of D-tartaric acid salt of (4aR,10aR)-1-ethyl-1,2,3,4,4a,5,6,7,8,9,10,10a-dodecahydrobenzo[g]quinolin-6-yl acetate

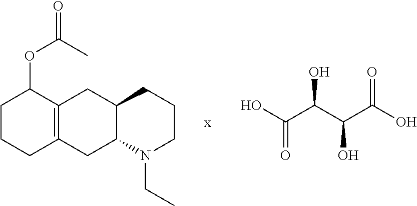 [SMILES: CCN1CCC[C@@H]2CC3=C(CCCC3OC(C)=O)C[C@H]21 ; O=C(O)[C@@H](O)[C@H](O)C(=O)O]

Epimer 1 of (4aR,10aR)-1-ethyl-1,2,3,4,4a,5,6,7,8,9,10,10a-dodecahydrobenzo[g]quinolin-6-ol, obtained in Preparation 1 (77 mg, 0.33 mmol), was dissolved in pyridine (1 mL) and to the solution was added acetic anhydride (1 mL, 10.6 mmol). The reaction mixture was stirred at room temperature overnight and then diluted with EtOH (10 mL). After stirring at room temperature for 30 minutes, the mixture was concentrated and then co-evaporated from EtOH several times until constant weight (91 mg). The weakly red oily residue was dissolved in EtOAc (8 mL). D-(−)-Tartaric acid (50 mg, 0.33 mmol) was added, and the mixture was stirred and heated at 50° C. for about 30 minutes in order to get a solution. Some insoluble dark-coloured material was removed with a spatula and the resulting clear mixture was stirred at room temperature for two days without using a stopper. A white precipitate had then been formed which was isolated by filtration. The solid was washed with EtOAc and dried under reduced pressure giving 74 mg (53%) of the desired D-tartrate as a beige powder.

Melting point: 136.4° C. MS (ESI+) m/z 278 [M+H]⁺. ¹H NMR (800 MHz, DMSO-d₆) δ 5.14 (m, 1H), 3.99 (s, 2H), 3.23 (d, 1H), 3.11 (m, 1H), 2.89 (m, 1H), 2.7-2.8 (m, 1H), 2.72-2.64 (m, 1H), 2.37 (dd, 1H), 2.13 (t, 1H), 2.00 (s, 3H), 1.9-2.0 (m, 3H), 1.8-1.9 (m, 2H), 1.6-1.8 (m, 5H), 1.5-1.6 (m, 2H), 1.1-1.2 (m, 4H). The NMR spectrum showed a ratio of 1:1 between the above-identified ester and D-tartaric acid. Thus, in this example the D-tartaric acid salt of (4aR,10aR)-1-ethyl-1,2,3,4,4a,5,6,7,8,9,10,10a-dodecahydrobenzo[g]quinolin-6-yl acetate is provided as a combination of (4aR,10aR)-1-ethyl-1,2,3,4,4a,5,6,7,8,9,10,10a-dodecahydrobenzo[g]quinolin-6-yl acetate and D-tartaric acid taken in a ratio of 1:1.

### Example 14

### Synthesis of (4aR,6R,10aR)-1-propyl-1,2,3,4,4a,5,6,7,8,9,10,10a-dodecahydrobenzo[g]quinolin-6-yl hexanoate

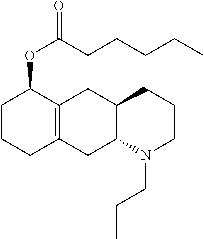 [SMILES: CCCCCC(=O)O[C@@H]1CCCC2=C1C[C@H]1CCCN(CCC)[C@@H]1C2]

Epimer 1 of (4aR,10aR)-1-propyl-1,2,3,4,4a,5,6,7,8,9,10,10a-dodecahydrobenzo[g]quinolin-6-ol, obtained in a similar fashion as in Example 4B (150 mg, 0.60 mmol), was dissolved in DCM (10 mL). Triethylamine (0.26 mL, 1.8 mmol) and hexanoic acid chloride (0.18 mL, 1.3 mmol) were added in the given order. The reaction mixture was stirred at room temperature for three days with the flask open to the air, and then the residue was dissolved in DCM. The formed solution was washed with aq. saturated NaHCO₃ (5 mL) and the volatiles were removed by evaporation. The product was purified by flash chromatography on silica gel using a gradient of 0 to 50% MeOH in EtOAc as eluent giving 66 mg (31%) of (4aR,10aR)-1-propyl-1,2,3,4,4a,5,6,7,8,9,10,10a-dodecahydrobenzo[g]quinolin-6-yl hexanoate in its non-salt form as an oil that darkened slowly. MS (ESI+) m/z 348 [M+H]⁺. ¹H NMR (600 MHz, CDCl₃) δ 5.23 (m, 1H), 3.08 (m, 1H), 2.73 (m, 1H), 2.47 (m, 1H), 2.1-2.4 (m, 5H), 1.5-2.0 (m, 14), 1.30 (m, 4H), 0.99 (m, 1H), 0.88 (m, 6H).

### Example 15

### Synthesis of D-tartaric acid salt of (4aR,10aR)-1-methyl-1,2,3,4,4a,5,6,7,8,9,10,10a-dodecahydrobenzo[g]quinolin-6-yl isobutyrate

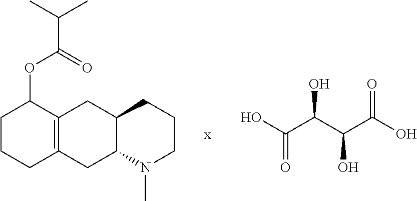 [SMILES: CC(C)C(=O)OC1CCCC2=C1C[C@H]1CCCN(C)[C@@H]1C2 ; O=C(O)[C@@H](O)[C@H](O)C(=O)O]

Epimer 1 of (4aR,10aR)-1-methyl-1,2,3,4,4a,5,6,7,8,9,10,10a-dodecahydrobenzo[g]quinolin-6-ol, obtained in Preparation 2 (100 mg, 0.45 mmol), was dissolved in DCM (4 mL) together with triethylamine (0.20 mL, 1.4 mmol). Isobutyryl chloride (0.1 mL, 0.95 mmol) was added to the formed solution and the mixture was stirred at room temperature overnight. Additional triethylamine (0.1 mL) and isobutyryl chloride (0.05 mL) were added and the mixture was stirred at room temperature for additional four hours. The mixture was washed with aq. saturated NaHCO₃ (5 mL) and the aq. phase was extracted with DCM. The combined organic layers were filtered through a phase separator and then concentrated. The product was purified by flash chromatography on silica gel using a gradient of 0 to 50% MeOH in EtOAc as eluent giving 82 mg of the ester as a brown oil. The residue was dissolved in EtOAc (7 mL). D-(−)-tartaric acid (41 mg, 0.27 mmol) was added, and the mixture was stirred and heated at 50° C. for about 30 minutes in order to get a clear solution. The mixture was stirred at room temperature with the flask open to the air leading to slow evaporation of the solvent. After two days, a few mL of solvent was still remaining, and a precipitate had been formed. The solid material was isolated by filtration, washed with EtOAc, and dried under reduced pressure giving 66 mg (55%) of the desired D-tartrate as a beige powder. Melting point: 97.3° C. MS (ESI+) m/z 292 [M+H]⁺. ¹H NMR (700 MHz, DMSO) δ 5.1-5.2 (m, 1H), 4.02 (s, 2H), 3.19 (d, 1H), 2.6 (m, 2H), 2.54 (s, 3H), 2.4-2.5 (m, 2H), 2.39 (dd, 1H), 2.0-2.2 (m, 1H), 1.9-2.0 (m, 3H), 1.6-1.8 (m, 7H), 1.4-1.6 (m, 2H), 1.0-1.1 (m, 7H). The NMR spectrum showed a ratio of 1:1 between the above-identified ester and D-tartaric acid. Thus, in this example the D-tartaric acid salt of_(4aR,10aR)-1-methyl-1,2,3,4,4a,5,6,7,8,9,10,10a-dodecahydrobenzo[g]quinolin-6-yl 2-isobutyrate is provided as a combination of (4aR,10aR)-1-methyl-1,2,3,4,4a,5,6,7,8,9,10,10a-dodecahydrobenzo[g]quinolin-6-yl 2-isobutyrate and D-tartaric acid taken in a ratio of 1:1.

### Example 16

### Crystallization Experiments in Relation to (4aS,6S,10aS)-1-propyl-1,2,3,4,4a,5,6,7,8,9,10,10a-dodecahydrobenzo[g]quinolin-6-yl acetate and (4aR,6R,10aR)-1-propyl-1,2,3,4,4a,5,6,7,8,9,10,10a-dodecahydrobenzo[g]quinolin-6-yl acetate, Respectively

The following experiments aimed at preparing crystalline salts of (4aS,6S,10aS)-1-propyl-1,2,3,4,4a,5,6,7,8,9,10,10a-dodecahydrobenzo[g]quinolin-6-yl acetate, which is the opposite enantiomer of (4aR,6R,10aR)-1-propyl-1,2,3,4,4a,5,6,7,8,9,10,10a-dodecahydrobenzo[g]quinolin-6-yl acetate (i.e. the compound of Formula IIIa1 as described herein and the non-salt form of the compound according to Example 6). The reason for using the opposite undesired enantiomer rather than the desired enantiomer (i.e. the compound of Formula IIIa1) was that the former isomer has no or very little effect on the dopamine receptors and thus the obtained material has no or little value in the research studies aimed at a dopamine receptor agonist. However, any chemical or physicochemical property found in a non-chiral environment such as solubility and crystallinity will be the same for the two enantiomers and thus any finding for the undesired enantiomer can be transferred to the desired enantiomer. Thus, Epimer 1 of (4aS,10aS)-1-propyl-1,2,3,4,4a,5,6,7,8,9,10,10a-dodecahydrobenzo[g]quinolin-6-ol—which was obtained in a similar fashion as in Example 4B but using the (+)-enantiomer of 1-propyl-1,2,3,4,4a,5,8,9,10,10a-decahydrobenzo[g]quinolin-6(7H)-one rather than the (−)-enantiomer of 1-propyl-1,2,3,4,4a,5,8,9,10,10a-decahydrobenzo[g]quinolin-6(7H)-one)—(1.1 g, 4.4 mmol) was dissolved in a mixture of pyridine (8 mL) and acetic anhydride (8 mL). The reaction mixture was placed in a fume hood overnight and then quenched by the addition of ethanol at room temperature. After 30 minutes, the volatiles were removed by evaporation and co-evaporation several times using ethanol. The residue was purified by silica-gel chromatography using a gradient of EtOAc and MeOH (0-70% MeOH) as eluent. There was obtained 0.78 g (61%) of (4aS,6S,10aS)-1-propyl-1,2,3,4,4a,5,6,7,8,9,10,10a-dodecahydrobenzo[g]quinolin-6-yl acetate in its non-salt form as an oil. The material was transferred into nine different vials (78 mg of the oil in each vial). Ethyl acetate (2 mL) was added to each of the vials together with one equivalent of one acid selected from nine different acids (namely maleic acid, oxalic acid, fumaric acid, succinic acid, L-tartaric acid (natural tartaric acid), benzoic acid, salicylic acid, benzenesulfonic acid and citric acid. Each vial was then equipped with a magnetic stirrer and each mixture was stirred at room temperature for a week without any precipitation being observed in any of the vials except in the vial comprising L-tartaric acid, which had provided a white precipitate after 24 h. The tartrate salt was isolated by filtration and the solid dried in the hood. There was obtained 86 mg (73%) of the L-tartrate salt as a white powder. Melting point: 83.4° C. [α]_{D}=+50.9° (c 10 mg/mL, MeOH). MS (ESI+) m/z 292 [M+H]⁺. The NMR spectrum showed a ratio of 1:1 between the above-identified ester and L-tartaric acid. Thus, in this example, the L-tartaric acid salt of (4aS,6S,10aS)-1-propyl-1,2,3,4,4a,5,6,7,8,9,10,10a-dodecahydrobenzo[g]quinolin-6-yl acetate is provided as a combination of (4aS,6S,10aS)-1-propyl-1,2,3,4,4a,5,6,7,8,9,10,10a-dodecahydrobenzo[g]quinolin-6-yl acetate and L-tartaric acid taken in a ratio of 1:1.

It is noteworthy to mention that attempts to perform the above experiment using (4aR,6R,10aR)-1-propyl-1,2,3,4,4a,5,6,7,8,9,10,10a-dodecahydrobenzo[g]quinolin-6-yl acetate and L-tartaric acid did not provide a solid salt but only a sticky oil. Instead, the only counterion that provided a crystalline salt together with this compound is the D-tartrate (which is the unnatural form of tartrate).

Thus, it was unexpectedly found that the non-natural form of tartaric acid, i.e. D-tartaric acid, allowed for forming a crystalline salt with (4aR,6R,10aR)-1-propyl-1,2,3,4,4a,5,6,7,8,9,10,10a-dodecahydrobenzo[g]quinolin-6-yl acetate. The D-tartaric acid salt of (4aR,6R,10aR)-1-propyl-1,2,3,4,4a,5,6,7,8,9,10,10a-dodecahydrobenzo[g]quinolin-6-yl acetate was a combination of D-tartaric acid and (4aR,6R,10aR)-1-propyl-1,2,3,4,4a,5,6,7,8,9,10,10a-dodecahydrobenzo[g]quinolin-6-yl acetate taken in a ratio of 1.1.

### Example 17

### Locomotor Activity

Behavioral activity was measured using eight Digiscan activity monitors (RXYZM (16) TAO, Omnitech Electronics, Columbus, OH, USA), connected to an Omnitech Digiscan analyzer and an Apple Macintosh computer equipped with a digital interface board (NB DIO-24, National Instruments, USA). Each activity monitor consisted of a quadratic metal frame (W×L=40 cm×40 cm) equipped with photo beam sensors. During measurements of behavioral activity, a rat was put in a transparent acrylic cage (W×L×H, 40×40×30 cm) which in turn was placed in the activity monitor. Each activity monitor was equipped with three rows of infrared photo beam sensors, each row consisting of 16 sensors. Two rows were placed along the front and the side of the floor of the cage, at a 90° angle, and the third row was placed 10 cm above the floor to measure vertical activity. Photo beam sensors were spaced 2.5 cm apart. Each activity monitor was fitted in an identical sound and light attenuating box containing a weak house light and a fan. The computer software was written using object-oriented programming (LabVIEW™, National instruments, Austin, TX, USA). Behavioral data from each activity monitor, representing the position (horizontal center of gravity and vertical activity) of the animal at each time, were recorded at a sampling frequency of 25 Hz and collected using a custom written LABView™ application. The data from each recording session were stored and analyzed with respect to distance traveled. Each behavioral recording session lasted 180 min, starting approximately 5 min after the injection of test compound.

Compounds disclosed herein have been tested for effects on spontaneous locomotor activity in non-pre-treated Sprague-Dawley rats (based on accumulated distance travelled 0-180 min post dosing), and with the single dose of 0.3 μmol/kg (n=5, SC) compared to a control group of animals (n=5) that obtained saline (SC). The prior art compound according to Preparation 4A was administered subcutaneously (SC). The compound according to Example 5 was administered perorally (PO) and subcutaneously (SC), respectively. The unit of the distance travelled is an arbitrary unit.

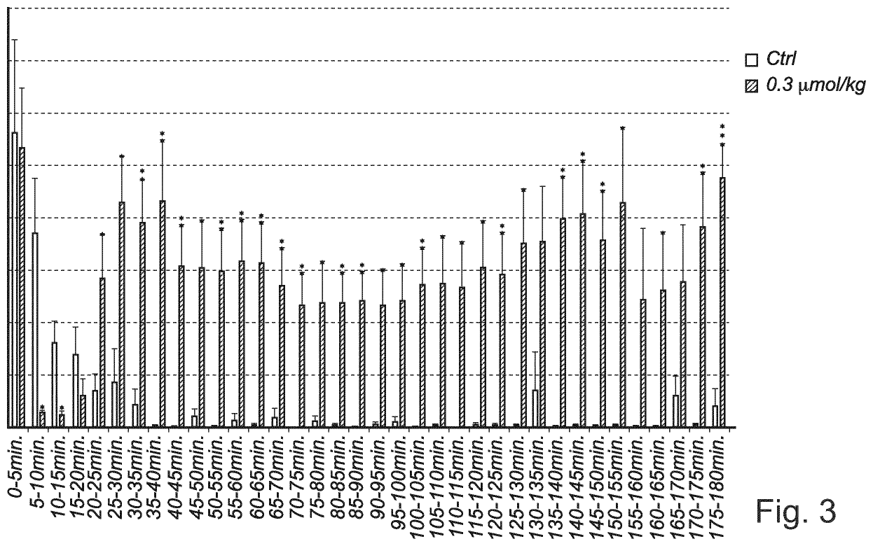

FIG. 3 shows the means of distance travelled after subcutaneous administration of either 0.3 μmol/kg of the prior art compound according to Preparation 4A ((4aR,10aR)-1-propyl-1,2,3,4,4a,5,8,9,10,10a-decahydrobenzo[g]quinolin-6(7H)-one) or administration of saline (control experiment) to drug-naïve rats. The animals were placed in the motility meters immediately after administration and locomotor activity was recorded for 180 minutes. Results are presented as distance travelled for the control group (empty bar) and for the group of animals that obtained the drug (filled bar).

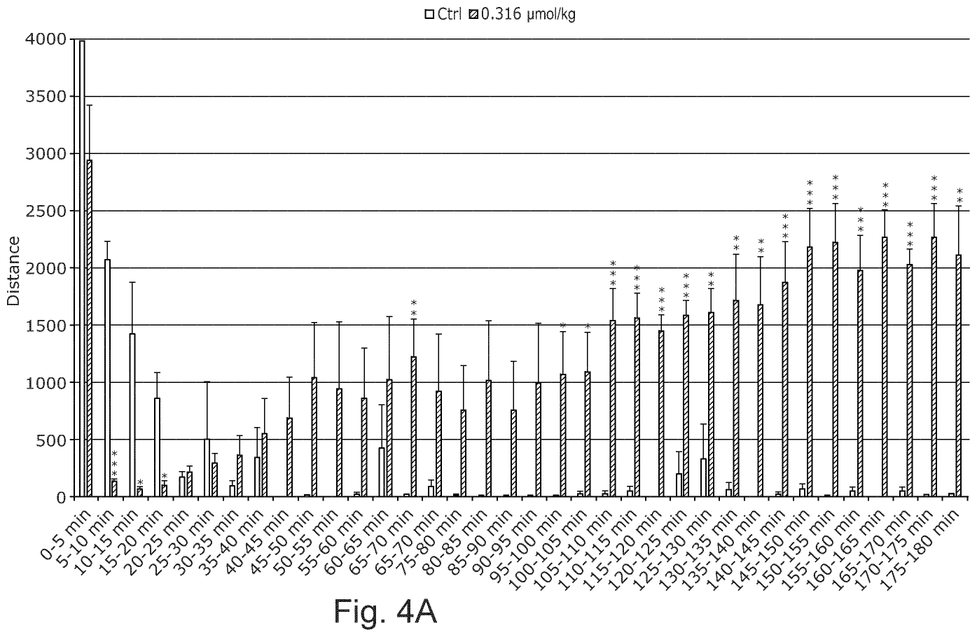

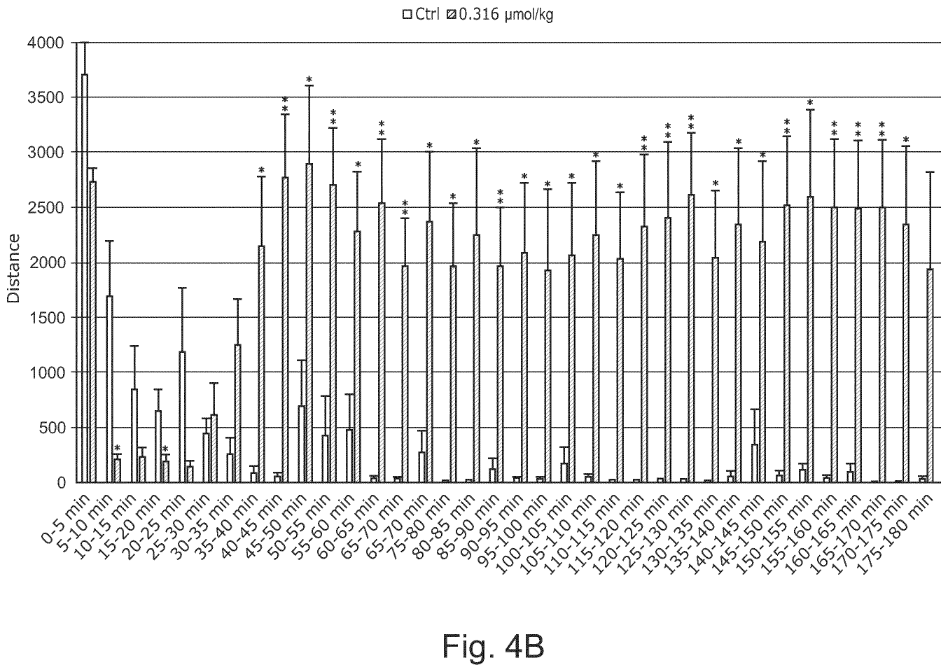

FIG. 4A shows the means of distance travelled after peroral administration of either 0.3 μmol/kg of the compound according to Example 5 (a mixture of the 6R and 6S epimers of oxalic acid salt of (4aR,10aR)-1-propyl-1,2,3,4,4a,5,6,7,8,9,10,10a-dodecahydrobenzo[g]quinolin-6-yl acetate) or peroral administration of saline (control experiment) to drug-naïve rats. The animals were placed in the motility meters immediately after administration and locomotor activity was recorded for 180 minutes. Results are presented as distance travelled for the control group (empty bars) and for the group of animals that obtained the drug (filled bar).

FIG. 4B shows the means of distance travelled after subcutaneous administration of either 0.3 μmol/kg of the compound according to Example 5 (a mixture of the 6R and 6S epimers of oxalic acid salt of (4aR,10aR)-1-propyl-1,2,3,4,4a,5,6,7,8,9,10,10a-dodecahydrobenzo[g]quinolin-6-yl acetate) or subcutaneous administration of saline (control experiment) to drug-naïve rats. The animals were placed in the motility meters immediately after administration and locomotor activity was recorded for 180 minutes. Results are presented as distance travelled for the control group (empty bars) and for the group of animals that obtained the drug (filled bar).

As shown in FIG. 3, FIG. 4A and FIG. 4B, both of the two compounds being tested do affect motor activity patterns in normal, non-pre-treated, rats. Thus, the prior art compound according to Preparation 4A as well as the compound according to Example 5 induce hyperactivity. The desired effect is lasting for at least 180 min for both of the two compounds showing that the two compounds do have a long duration of action. The trend appears to be that the compound according to Example 5 has a longer duration of action as compared to the prior art compound as an increase in the desired effect is observed for the compound according to Example 5 much later in the experiment. Furthermore, an even more pronounced difference between the two compounds is that the on-set of action, i.e. that the distance travelled increases after the initial decrease in distance travelled taking place immediately after administration, for the prior art compound is faster (20-25 min) as compared to the compound according to Example 5 (35-40 min). A further difference is that the immediate effect on motor activity for the prior art compound is much steeper with a long distance travelled already within 20-45 min. These data taken together show that the animals obtaining the prior art compound, quickly are having a higher peak plasma concentration of the active species resulting from the administered drug as compared to that of the compound according to Example 5. Consequently, the data show that the compound according to Example 5 is associated with no or a mild side effect profile due to a slower on-set of action.

### Example 18

m-RNA Analysis:

Animals were killed 60 min after the injection of the drugs by decapitation.

The brains were dissected into a left and a right part. The left part was analyzed for gene expression and dissected into 4 different areas:

Limbic system (containing nucleus accumbens, most parts of the olfactory tubercle, ventral pallidum and amygdala), striatum, frontal cortex, and hippocampus.

Total RNA was prepared by RNeasy Plus Universal Tissue Mini Kit (Qiagen).

RNA pellets were dissolved in RNAse-free water and stored at −80° C. The sample concentration was determined spectrophotometrically by a Nanorop ND-1000.

A two-step reversed transcription was performed by using a SuperScript III kit (Invitrogen). 1 μg of total RNA was reversed transcribed with 5 μl 2× RT Reaction Mix, 1 μl RT Enzyme and the mix volume was adjusted to 10 μl with RNAse-free water. The samples were incubated at 25° C. for 10 mi, 50° C. for 30 mi and finally 85° C. for 5 mi. 1 U of E. coli RNase H was added following incubation at 37° C. for 20 mi and 85° C. for 5 minutes. The cDNA solution was diluted 40 times in Tris EDTA buffer solution pH8 (Merck) and stored at −20° C.

Three sequences (arc and two reference genes) were amplified together in a triplex PCR-reaction. For real-time PCR measurements: 5 μl of the cDNA reaction was amplified in a 20 μl reaction mixture containing 10 μl PerfeCTa MultiPlex qPCR SuperMix (Quantabio), 3.5 μl RNAse-free water, 0.15 μM of each primer and 0.1 μM of each probe. Real-time PCR was measured on CFX96 (Bio-Rad) using the following settings for all genes: 3 mi pre-incubation at 95° C. followed by 40 cycles of denaturation at 95° C. for 15s, annealing and elongation at 60° C. for 1 mi. Reference genes are HPRT and cyclophilin.

### Tagman Single and Duplex PCR for Analysis of EGR-1 and Npas4

The real-time PCR reaction consisted of 10 μl Sso Advanced Universal Probes Supermix, 1 μl primer/probe, 1 μl reference gene or 1 μl MQ water and 8 μl of cDNA (diluted 40 times from RT-PCR). Real-time PCR reactions were performed in a CFX96 Real-Time PCR Detector (Bio-Rad) with the following cycling conditions: initial denaturation at 95° C. for 2 mi followed by 40 cycles of 95° C. for 5 s and 60° C. for 30 s. All genes of interest were labelled with the fluorophore FAM on the 5 end and reference genes (HPRT and ppia (also named cyclophilin)) were labelled with HEX. TaqMan primers and probes were synthesized by Bio-rad (Coralville, Iowa, USA) and used according to the manufacturing protocol.

EGR-1 (Early growth response qRnoCEP0022872) was analyzed in duplex with the reference gene HPRT (hypoxanthine phosphoribosyltransferase qRnoCEP0050840). Npas4 (neuronal PAS domain protein4 qRnoCEP0029461) was analyzed in singleplex. The reference gene Ppia (cyclophilin A peptidyl-propyl cis-trans isomeraseqRnoCIP0050815) was also analyzed in order to quantify gene expression for genes of interest.

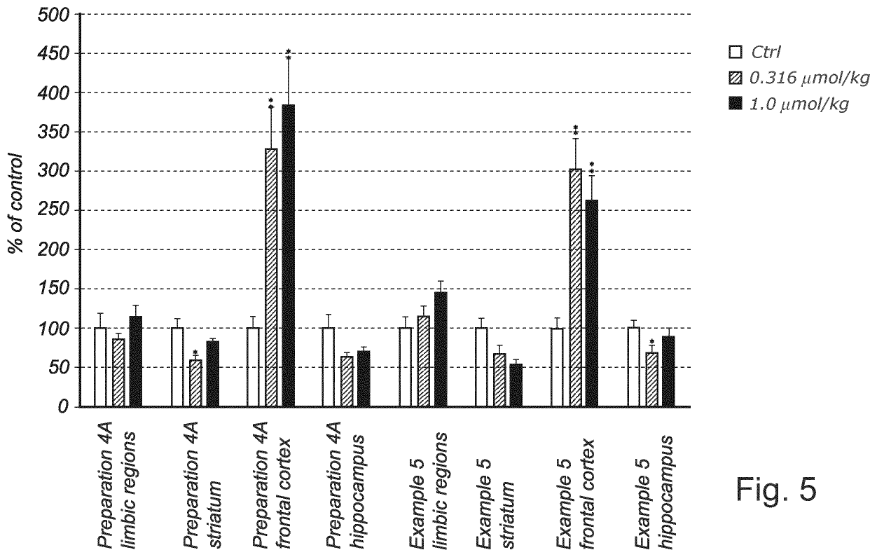

FIG. 5 illustrates the effects on tissue levels of Arc mRNA in four different regions of the brain (limbic regions, striatum, frontal cortex and hippocampus) after subcutaneous administration of two different compounds at two different doses as in comparison to that of corresponding control experiments. The bars in the left half of the figure represent the effects on ARC by the prior art compound according to Preparation 4A ((4aR,10aR)-1-propyl-1,2,3,4,4a,5,8,9,10,10a-decahydrobenzo[g]quinolin-6(7H)-one) and the bars in the right half of the figure represent the effects on ARC by the compound of the present disclosure according to Example 5 oxalic acid salt of ((4aR,10aR)-1-propyl-1,2,3,4,4a,5,6,7,8,9,10,10a-dodecahydrobenzo[g]quinolin-6-yl acetate (Mixture of 6R and 6S epimers). The effects on tissue levels of Arc by both of the two aforementioned compounds were measured at two different doses (0.3 μmol/kg and 1 μmol/kg) and the effect is presented as percent of control means±SEM. Statistical significance was assessed using Student's t-test (2 tailed) vs controls.

As shown in the diagram of FIG. 5, both of the two compounds dose-dependently increase tissue levels of Arc in the frontal cortex, which sometimes is observed for a dopamine receptor agonist. Also shown in the diagram is that the compound according to Example 5 dose-dependently increases the tissue levels of Arc in the limbic regions which is a property that the prior art compound according to Preparation 4A does not have. As Arc is a biomarker of synaptic activity, this attribute of the compound according to Example 5 allows for providing a unique therapeutic profile such as improvements related to emotion, behavior, and/or long-term memory. Furthermore, the compound according to Example 5 increases tissue levels of other genes in the limbic regions such as for instance Npas4. The compound does so to a greater extent as compared to that of the prior art compound according to Preparation 4A and this observed effect allows for an improved therapy for the patients with neurodegenerative diseases and/or neurological disorders.

### Sequence Listing

The primer and probe sequences are as follows for measuring of arc:

### Activity-Regulated Gene Arc Accession Number U19866

Sense:
(SEQ ID NO: 1)
5′- GGA GTT CAA GAA GGA GTT TC-3′
Antisense:
(SEQ ID NO: 2)
5′- CCA CAT ACA GTG TCT GGT A -3′
Probe:
(SEQ ID NO: 3)
CCG CTT ACG CCA GAG GAA CT

Dye: 5′FAM Quencher: 3′BHQ1
Product size: 149

### Hypoxantine Phosphoribosyl Transferase (HPRT) (Accession Number AF001282)

Sense:
(SEQ ID NO: 4)
5′- AGG GAT TTG AAT CAT GTT TG -3′
Antisense:
(SEQ ID NO: 5)
5′- CTG CTA GTT CTT TAC TGG C -3′
Probe:
(SEQ ID NO: 6)
TGT AGA TTC AAC TTG CCG CTG TC

Dye: 5′HEX Quencher: 3′BHQ1
Product size: 121

### Cyclophilin a (Cyclo) (Accession Number M19533)

Sense:
(SEQ ID NO: 7)
5′- CTG GAC CAA ACA CAA ATG-3′
Antisense:
(SEQ ID NO: 8)
5′- ATG CCT TCT TTC ACC TTC -3′
Probe:
(SEQ ID NO: 9)
TTG CCA TCC AGC CAC TCA GT

Dye: 5′Texas red Quencher: 3′BHQ2
Product size: 100

### REFERENCES

1. Liu et al., “Extremely Potent Orally Active Benzo[g]quinoline Analogue of the Dopaminergic Prodrug: 6-(N,N-Di-n-propyl)amino-3,4,5,6,7,8-hexahydro-2H-naphtalen-1-one”, J. Med. Chem., 2006, 49, 1494-1498 (The title of this article was soon afterwards corrected to “Extremely Potent Orally Active Benzo[g]quinoline Analogue of the Dopaminergic Prodrug: 1-Propyl-trans-2,3,4,4a,5,7,8,9,10,10a-decahydro-1H-benzo[g]quinoline-6-one”, J. Med. Chem., 2006, 49, 6930)
2. Liu et al., “A novel synthesis and pharmacological evaluation of a potential dopamine D1/D2 agonist: 1-Propyl 1,2,3,4,4a,5,10,10a-octahydrobenzo[g]quinoline-6,7-diol “Bioorganic & Medicinal Chemistry, 16 (2008), 3438-3444
3. WO 2010/097092
4. WO 2001/078713
5. WO 2019/101917
6. WO 2020/234270
7. WO 2020/234271
8. WO 2020/234272
9. WO 2020/234273
10. WO 2020/234274
11. WO 2020/234275
12. WO 2020/234276
13. WO 2020/234277

1. A compound of Formula III1: 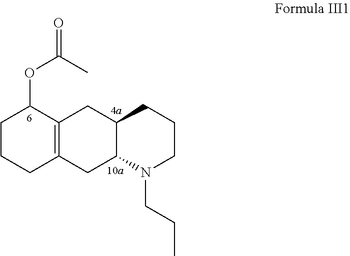 [SMILES: CCCN1CCC[C@@H]2CC3=C(CCCC3OC(C)=O)C[C@H]21]
or a pharmaceutically acceptable salt thereof,
wherein
carbon 4a and carbon 10a both have R configuration.

2. The compound according to claim 1, wherein carbon 6 has R configuration thereby providing a compound of Formula IIIa1: 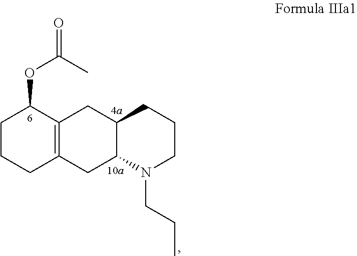 [SMILES: CCCN1CCC[C@@H]2CC3=C(CCC[C@H]3OC(C)=O)C[C@H]21]
or a pharmaceutically acceptable salt thereof.

3. The compound according to claim 1, wherein carbon 6 has S configuration thereby providing a compound of Formula IIIb1: 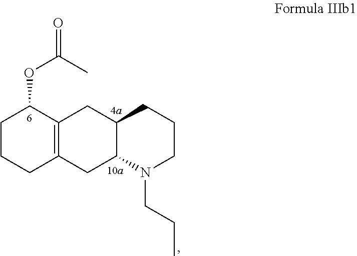 [SMILES: CCCN1CCC[C@@H]2CC3=C(CCC[C@@H]3OC(C)=O)C[C@H]21]
or a pharmaceutically acceptable salt thereof.

4.-8. (canceled)

9. A pharmaceutically acceptable salt of the compound according to claim 1.

10. The pharmaceutically acceptable salt according to claim 9, wherein said pharmaceutically acceptable salt is a combination of the compound of Formula III1 and an organic acid.

11. The pharmaceutically acceptable salt according to claim 10, wherein said pharmaceutically acceptable salt is a combination of the compound of Formula III1 and said organic acid in a ratio of 2:1.

12. The pharmaceutically acceptable salt according to claim 10, wherein said pharmaceutically acceptable salt is a combination of the compound of Formula III1 and said organic acid in a ratio of 1:1.

13. The pharmaceutically acceptable salt according to claim 10, wherein said organic acid is D-tartaric acid.

14. The compound according to claim 1, or a pharmaceutically acceptable salt thereof wherein the compound is in solid form.

15. The compound according to claim 1, or a pharmaceutically acceptable salt thereof, wherein the compound is in crystalline form.

16. A pharmaceutical composition comprising a therapeutically acceptable amount of the compound according to claim 1, or a pharmaceutically acceptable salt thereof, in admixture with at least one pharmaceutically acceptable carrier, excipient and/or diluent.

17.-29. (canceled)

30. A method for treatment of one or more of the following:
Parkinson's disease, Huntington's disease, Restless leg syndrome, Alzheimer's disease, schizophrenia, attention deficit hyperactivity disorder, and drug addiction, said method comprising administering to a mammal in need thereof, a therapeutically effective amount of:
the compound according to claim 1, or a pharmaceutically acceptable salt thereof.

31. The method according to claim 30, wherein the mammal is a human.

32. The method according to claim 30, wherein the treatment comprises treatment of Parkinson's disease.

33. The method according to claim 30, wherein the treatment is associated with no side effect(s) or mild side effect(s) comprising nausea and/or vomiting.

34. (canceled)

35. The method according to claim 32, wherein the treatment further comprises treatment of non-motor symptom(s) associated with Parkinson's disease.

36. The method according to claim 35, wherein the non-motor symptom(s) associated with Parkinson's disease comprise(s) one or more of the following: cognitive functional decline, depression, anxiety, apathy, and Parkinson's disease dementia.

37. A method for preparing a compound of Formula III1 according to claim 1, or a pharmaceutically acceptable salt thereof,
said method comprising the steps of:
a) reacting a compound of Formula II1 with a compound of Formula IV 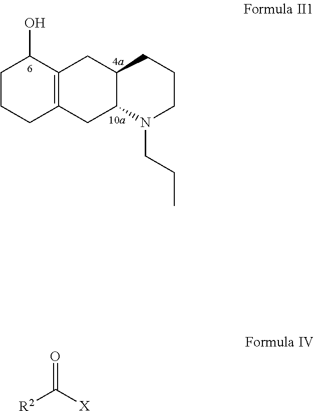
wherein
carbon 4a and carbon 10a in the compound of Formula II both have R configuration, X is selected from the group consisting of OH, halide and OC(O)R², and
R² is methyl,
optionally in the presence of an ester forming promoting agent such as a coupling reagent
thereby forming the compound of Formula III1,
b) optionally separating the compound of Formula III1, into a compound of Formula IIIa1 and a compound of Formula IIIb1, and
c) optionally combining the compound of Formula III1 obtained in step a) or step b) with a pharmaceutically acceptable acid thereby providing a pharmaceutically acceptable salt of the compound of Formula III1.

38. (canceled)

39. The method according to claim 37, wherein the compound of Formula II1 is prepared by a process comprising the steps of:
a) reducing the compound of Formula I 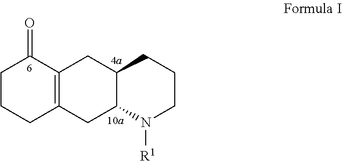
wherein R¹ is n-propyl,
with a reducing agent in the presence of a Lewis acid and a solvent
whereby the carbonyl group is reduced into a hydroxyl group thereby providing the compound of Formula II1, and
b) optionally separating the compound of Formula II1 into a compound of Formula IIa1 and Formula IIb1

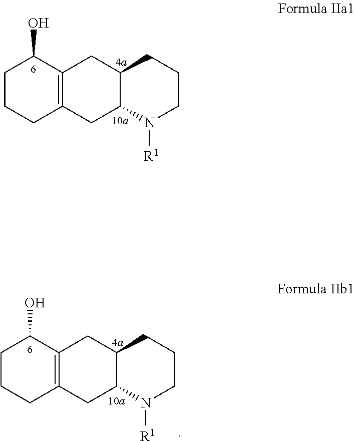

40.-41. (canceled)

42. The pharmaceutically acceptable salt according to claim 9, wherein said pharmaceutically acceptable salt is a combination of the compound of Formula IIIa1 and D-tartaric acid in a ratio of 1:1.

43. The pharmaceutically acceptable salt according to claim 11, wherein said organic acid is D-tartaric acid.
